(19) United States
(12) Patent Application Publication
(10) Pub. No.: US 2026/0043014 A1
(43) Pub. Date: Feb. 12, 2026

(54) STABILIZED PROTEIN PRODUCTION PROCESS USING BACILLUS HOST CELLS WITH SALT FEED

(71) Applicant: BASE SE, Ludwigshafen am Rhein (DE)

(72) Inventors: Stefan JENEWEIN, Ludwigshafen am Rhein (DE); Andreas DAUB, Ludwigshafen am Rhein (DE); Tobias KLEIN, Ludwigshafen am Rhein (DE); Georg Benjamin WANDREY, Ludwigshafen am Rhein (DE); Michael MORWEISER, Ludwigshafen am Rhein (DE)

(21) Appl. No.: 19/100,485

(22) PCT Filed: Aug. 1, 2023
(86) PCT No.: PCT/EP2023/071318

§ 371 Date: Jan. 31, 2025

(30) Foreign Application Priority Data
Aug. 2, 2022 (EP) 22188310.1

(51) Int. Cl. C12N 9/54; C12P 21/02

(52) U.S. Cl. CPC C12N 9/54; C12P 21/02; C12Y 304/21063

(57) ABSTRACT

The present invention relates to the field of industrial fermentation and protein production. In particular, it relates to a method for producing a protein of interest in a fermentation medium comprising the following steps a) inoculating a fermentation medium with a Bacillus host cell comprising a gene encoding a protein of interest under the control of a promoter; b) cultivating the Bacillus host cell in the fermentation medium under conditions conducive for the growth of the Bacillus host cell and the expression of the protein of interest, c) adding sulfate to the fermentation medium to reach a concentration of at least 20 mM of sulfate in the fermentation medium; and d) allowing the protein of interest to precipitate and/or crystallize during cultivation. Further contemplated is the use of a high sulfate concentration in a fermentation medium for producing a protein of interest in a Bacillus host cell and a crystallized protein of interest obtained by or obtainable by the method of the invention.

The present invention relates to the field of industrial fermentation and protein production. In particular, it relates to a method for producing a protein of interest in a fermentation medium comprising the following steps a) inoculating a fermentation medium with a Bacillus host cell comprising a gene encoding a protein of interest under the control of a promoter; b) cultivating the Bacillus host cell in the fermentation medium under conditions conducive for the growth of the Bacillus host cell and the expression of the protein of interest, c) adding sulfate to the fermentation medium to reach a concentration of at least 20 mM of sulfate in the fermentation medium; and d) allowing the protein of interest to precipitate and/or crystallize during cultivation.

Further contemplated is the use of a high sulfate concentration in a fermentation medium for producing a protein of interest in Bacillus host cell and a crystallized protein of interest obtained by or obtainable by the method of the invention.

Microorganisms are widely used as industrial workhorses for the production of a protein of interest, in particular an enzyme. The biotechnological production of a protein of interest is conducted via a fermentation process involving cultivation of the microorganism and subsequent purification of the product.

Microorganisms, like Bacillus species, are capable of secreting significant amounts of protein of interest into the fermentation medium. This allows a more simple purification of the protein of interest compared to intracellular production and explains the success of Bacillus host cells in industrial applications. Moreover, high titers of the protein of interest can be achieved and are also desired. Recent progress in strain and fermentation methodology has led to an increase in titers of proteins of interest.

However, high titers of the protein of interest can also have disadvantages. For example, high titers of the protein of interest may lead to undesired protein instability and other undesired effects on the protein of interest and/or the host organism, such as irreversible aggregation, inactivation and/or (auto) proteolytic processes, that may reduce the fitness of the host or may reduce the yield of the protein of interest already during cultivation and in subsequent purification processes.

Different approaches have been reported aiming at increasing the yield of protein of interest produced by the Bacillus cells and stabilizing the protein of interest during said cultivation in large scale bioreactors. These approaches concerned, e.g., variations in the composition of media. Other approaches concerned a decrease in temperature, inter alia, for reducing the likelihood of inclusion body formation (Hashemi 2012, Food Bioprocess Technol 5:1093-1099; Wenzel 2011, Applied and Environmental Microbiology 77: 6419-6425).

WO93/13125 describes a protein production process of a protein susceptible to inactivation and degradation during fermentation. The process aims at producing a protease variant carrying particular amino acid substitutions in a Bacillus subtilis strain by continuously and reversibly protecting said protease against said inactivation or degradation during the production stage. The protection is largely achieved by reducing solubility of the protein of interest by genetic engineering, i.e. introducing said particular amino acid substitutions in the protease sequence. The protease precipitates in the fermentation medium and is then separated and purified while for other commonly used proteases, e.g. non-engineered proteases, no precipitation occurs. Hence, this protein production process does not present a solution for the production of enzymes that are prone to (auto) hydrolysis and that do not carry substitutions resulting in protective precipitation.

WO 2018/185048 discloses a recovery process for recovering a protein of interest that has partially precipitated during fermentation. The recovery process involves several steps including a first separation step, followed by a second solubilization step.

Similarly U.S. Pat. No. 6,316,240 deals with recovering a glycosidase or a peptidase from a culture medium, from an insoluble state. A method is disclosed comprising suspending the solid fraction of a fermentation medium and adjusting the pH to allow for solubilization and recovery of the protein of interest. A further process for recovering a protein of interest from an insoluble state is disclosed in EP 2125865 that describes a method for solubilizing protease crystals or protease precipitate in a fermentation broth.

Although several processes for recovering protein crystals and precipitates have been described, there is still a further need for achieving a simple and stable production of native or wild-type enzymes that have not been engineered for precipitation or crystal formation but are prone to (auto) hydrolysis or that are otherwise instable. The precipitation is required to work at fermentation conditions preferably at conditions that are conducive for the growth of the Bacillus host cell and the expression of the protein of interest, typically conditions where the viability of the Bacillus host cell is not affected. Most preferably the precipitation is performed at the optimal or close to the optimal growth condition of the Bacillus host cell. Relevant parameters for optimal growth are for example pH, salt or temperature and are known to the person skilled in the art.

Means for further increasing yield and stabilizing the protein of interest in large-scale industrial fermentation processes are hence still highly desired.

The technical problem underlying the present invention may be regarded as the provision of means and methods for complying with the aforementioned needs. The technical problem is solved by the embodiments characterized in the claims and herein below.

Thus, the present invention relates to a method for producing a protein of interest in a fermentation medium comprising the following steps a) inoculating a fermentation medium with a Bacillus host cell comprising a gene encoding a protein of interest under the control of a promoter; b) cultivating the Bacillus host cell in the fermentation medium under conditions conducive for the growth of the Bacillus host cell and the expression of the protein of interest, c) adding sulfate to the fermentation medium to reach a concentration of at least 20 mM of sulfate in the fermentation medium; and d) allowing the protein of interest to precipitate and/or crystallize during cultivation; wherein the fermentation medium comprises an amino acid derivative in an amount of 0-30 g/l of fermentation medium.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

It is to be understood that as used in the specification and in the claims, “a” or “an” can mean one or more, depending upon the context in which it is used. Thus, for example, reference to “a host cell” can mean that at least one host cell can be utilized.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. The term “comprising” as used herein shall not be understood in a limiting sense. The term rather indicates that more than the actual items referred to may be present, e.g., if it refers to a method comprising certain steps, the presence of further steps shall not be excluded. However, the term also encompasses embodiments where only the items referred to are present, i.e. it has a limiting meaning in the sense of “consisting of”.

Further, as used in the following, the terms “particularly”, “more particularly”, “typically”, and “more typically” or similar terms are used in conjunction with additional or alternative features, without restricting alternative possibilities. Thus, features introduced by these terms are additional or alternative features and are not intended to restrict the scope of the claims in any way. The invention may, as the skilled person will recognize, be performed by using alternative features. Similarly, features introduced by “in an embodiment of the invention” or similar expressions are intended to be additional or alternative features, without any restriction regarding alternative embodiments of the invention, without any restrictions regarding the scope of the invention and without any restriction regarding the possibility of combining the features introduced in such way with other additional or alternative or non-additional or alternative features of the invention. Not mandatory, but preferred features are also characterized by the term “preferably”, “more preferably” or “most preferably”.

Further, it will be understood that the term “at least” means that the item or parameter to which the term refers is limited in one direction but open ended in one or more other directions. Furthermore, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, or even more preferably ±5%.

The present invention, thus, provides for a method that can be applied for culturing Bacillus host cells in both, laboratory and industrial scale fermentation processes. “Industrial fermentation” as referred to in accordance with the present invention refers to a cultivation method in which at least 100 g of a carbon source per kg of initial fermentation medium will be added, preferably at least 200 g of a carbon source per kg of initial fermentation medium will be added.

Such large-scale fermentation processes usually comprise growing the inoculated Bacillus host cells under conditions which allow for growth and expression of the protein of interest to be produced. Typically, host cells of a suitable microorganism such as Bacillus host cells are grown in complex or defined fermentation media and carbon sources will be fed in constant or varying amounts during cultivation. More typically, in line with the present invention, Bacillus host cells are grown in a chemically defined medium as specified elsewhere herein.

The method according to the present invention comprises at least the following steps: a) inoculating a fermentation medium with a Bacillus host cell comprising a gene encoding a protein of interest under the control of a promoter; b) cultivating the Bacillus host cell in the fermentation medium under conditions conducive for the growth of the Bacillus host cell and the expression of the protein of interest, c) adding sulfate to the fermentation medium to reach a concentration of at least 20 mM of sulfate in the fermentation medium; and d) allowing the protein of interest to precipitate and/or crystallize during cultivation.

Optionally, the method may comprise a step of adding an amino acid derivative to the fermentation medium. Preferably, the method according to the invention does not comprise the addition of an amino acid derivative to the fermentation medium.

The steps, as outlined above, may typically be performed in the given order or, in any other possible order, or in parallel.

It is understood that in particular steps b), c), d) and the optional step of adding an amino acid derivative can be done sequentially in the given order or in any other possible order, or can be done in parallel. Typically, said steps can be done in parallel in case when the initial fermentation media already contains sulfate and/or the amino acid derivative is already added. Alternatively, step b) can be combined with the step of adding an amino acid derivative and the step of sulfate addition may be performed thereafter. Still alternatively, step c) and the step of adding an amino acid derivative can be done in parallel.

In the method according to the invention preferably the desired concentration of sulfate is maintained throughout the fermentation process. Preferably, sulfate is added to the process in the desired concentration at an early stage, preferably as early as the production of the protein of interest starts, or even before. Reaching the desired high concentration of sulfate early during the fermentation process is advantageous as the desired concentration of sulfate results in an early and immediate efficient protection from proteolysis. Without wishing to be bound by theory, the present inventors believe this protection from proteolysis is due to the precipitation of the protein of interest. Adding sulfate to the fermentation medium may particularly refer to adding sulfate with the feed to reach and/or to maintain a concentration of at least 20 mM of sulfate in the fermentation medium. Higher concentrations of sulfate may be desired and defined elsewhere herein.

According to the invention, the desired high level of precipitant, in particular sulfate, may be achieved as early as possible and preferably is maintained during the fermentation, more preferably with start of the production phase of the protein of interest. This is advantageous as it enables an early precipitation and hence a protection from proteolysis. More preferably, the desired concentration of precipitant is present from the start of the cultivation such as in the initial fermentation medium.

The “start of the production phase of the protein of interest” or “start of the production of the protein of interest” refers to the point in time during the main fermentation when the protein of interest can be detected first, in other words the point in time when protein production starts. The start of the production of the protein of interest can be detected by detecting the protein of interest. The detection of the protein of interest may typically be performed by assaying for the presence of protein activity using standardized tests known to the person of skill in the art and depending on the type of the protein of interest. Typically, these are tests for assaying enzymatic activity if the protein of interest is an enzyme. In the event that the protein of interest is protease, the test is a test for assaying proteolytic activity, such as assaying substrate cleavage resulting in a detectable product, such as colorimetric or fluorescent product. Such assays are commonly known to the person of skill in the art and often even commercially available. For example, protease activity may be determined by using the commercially available protease substrate Succinyl-Ala-Ala-Pro-Phe-p-nitroanilide (Suc-AAPF-pNA, short AAPF; see e.g. DelMar et al. (1979), Analytical Biochem 99, 316-320). pNA is cleaved from the substrate molecule by the activity of the protease, in particular by proteolytic cleavage at 30° C. and at pH 8.6 in TRIS (2-Amino-2-(hydroxymethyl) propane-1,3-diol) buffer. The protease activity results in release of free pNA from said substrate. Free pNA has a yellow color and that may be quantified by measuring OD405.

The method according to the present invention may also comprise further steps. Such further steps may encompass the termination of cultivating and/or obtaining a product such as the protein of interest from the Bacillus host cell culture by appropriate purification techniques. Preferably, the method of the invention further comprises the step of obtaining the protein of interest from the Bacillus host cell culture obtained after cultivation, typically at the end of the fermentation process.

In the method of the present invention, a fermentation medium is inoculated with a Bacillus host cell comprising a gene encoding a protein of interest under the control of a promoter, typically as a first step, i.e. at the beginning of the fermentation process.

The term “Bacillus host cell” refers to a Bacillus cell which serves as a host for an expression construct for a gene encoding a protein of interest. Said expression construct may be a naturally occurring expression construct, a recombinantly introduced expression construct or a naturally occurring expression construct which has been genetically modified in the Bacillus cell. The Bacillus host cell may be a host cell from any member of the bacterial genus Bacillus, preferably a host cell of Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Geobacillus stearothermophilus, Bacillus thuringiensis or Bacillus velezensis. More preferably, the Bacillus host cell is a cell of Bacillus amyloliquefaciens, Bacillus clausii, Bacillus haloduans, Bacillus lentus, Bacillus licheniformis, Bacillus pumilus, Bacillus subtilis, or Bacillus velezensis. Still more preferably, the Bacillus host cell is a Bacillus licheniformis, Bacillus pumilus, or Bacillus subtilis host cell, even more preferred Bacillus licheniformis or Bacillus subtilis host cell, most preferably, Bacillus licheniformis host cell. Particular preferably, the Bacillus licheniformis is selected from the group consisting of Bacillus licheniformis as deposited under American Type Culture Collection number ATCC 14580, ATCC 31972, ATCC 53926, ATCC 53757, ATCC 55768, and under DSMZ number (German Collection of Microorganisms and Cell Cultures GmbH) DSM 13, DSM 394, DSM 641, DSM 1913, DSM 11259, and DSM 26543.

Typically, the host cell belongs to the species Bacillus licheniformis, such as a host cell of the Bacillus licheniformis strain ATCC 14580 (which is the same as DSM 13, see Veith et al. “The complete genome sequence of Bacillus licheniformis DSM 13, an organism with great industrial potential.” J. Mol. Microbiol. Biotechnol. (2004) 7:204-211). Alternatively, the host cell may be a host cell of Bacillus licheniformis strain ATCC 53926. Alternatively, the host cell may be a host cell of Bacillus licheniformis strain ATCC 31972. Alternatively, the host cell may be a host cell of Bacillus licheniformis strain ATCC 53757. Alternatively, the host cell may be a host cell of Bacillus licheniformis strain ATCC 53926. Alternatively, the host cell may be a host cell of Bacillus licheniformis strain ATCC 55768. Alternatively, the host cell may be a host cell of Bacillus licheniformis strain DSM 394. Alternatively, the host cell may be a host cell of Bacillus licheniformis strain DSM 641. Alternatively, the host cell may be a host cell of Bacillus licheniformis strain DSM 1913. Alternatively, the host cell may be a host cell of Bacillus licheniformis strain DSM 11259. Alternatively, the host cell may be a host cell of Bacillus licheniformis strain DSM 26543.

The Bacillus host cell to be applied in the method of the present invention shall comprise an expression construct for a gene encoding a protein of interest to be expressed by the said host cell. The term “expression construct” as referred to herein refers to a polynucleotide comprising a nucleic acid sequence, e.g. a gene, encoding the protein of interest operably linked to an expression control sequence, e.g., a promoter. Typically, the expression construct as used in the method according to the invention may at least comprise a nucleic acid sequence encoding the protein of interest operably linked to a promoter.

A promoter as referred to herein is a nucleotide sequence located upstream of a gene on the same strand as the gene that enables transcription of said gene. The activity of a promoter (also referred to as promoter activity) is understood herein as the capacity of the promoter to enable and initiate transcription of said gene, in other words it is understood as the capacity of the promoter to drive gene expression. The promoter is followed by the transcription start site of the gene. The promoter is recognized by an RNA polymerase, typically, together with the required transcription factors, which initiate transcription. A functional fragment or functional variant of a promoter is a nucleotide sequence which is recognizable by RNA polymerase and is capable of initiating transcription. Functional fragments or functional variants of promoters are also encompassed as a promoter in the sense of the present invention.

Promoters may be inducer-dependent promoters the activity of which depend on an activating signal molecule, i.e., the presence of an inducer molecule, or may be inducer-independent promoters, i.e. promoters that do not depend on the presence of an inducer molecule added to the fermentation medium and that are either constitutively active or can be increased in activity regardless of the presence of an inducer molecule that is added to the fermentation medium. Preferably, the promoter is an inducer-independent promoter.

Preferably, the promoter is selected from the group consisting of the promoter sequences of the aprE promoter (a native promoter from the gene encoding the Bacillus subtilisin Carlsberg protease), amyQ promoter from Bacillus amyloliquefaciens, amyL promoter and variants thereof from Bacillus licheniformis (preferably as de-scribed in U.S. Pat. No. 5,698,415), bacteriophage SPO1 promoter, such as the promoter PE4, PE5, or P15 (preferably as described in WO2015118126 or in Stewart, C. R., Gaslightwala, I., Hinata, K., Krolikowski, K. A., Needleman, D. S., Peng, A. S., Peterman, M. A., Tobias, A., and Wei, P. 1998, Genes and regulatory sites of the “host-takeover module” in the terminal redundancy of Bacillus subtilis bacteriophage SPO1. Virology 246(2), 329-340), cryIIIA promoter from Bacillus thuringiensis (preferably as described in WO9425612 or in Agaisse, H. and Lereclus, D. 1994. Structural and functional analysis of the promoter region involved in full expression of the cryIIIA toxin gene of Bacillus thuringiensis. Mol. Microbiol. 13(1). 97-107.), and combinations thereof, and active fragments or variants thereof.

Preferably, the promoter sequences can be combined with 5′-UTR sequences native or heterologous to the host cell, as described herein. Preferably, the promoter is an inducer-independent promoter. More preferably, the promoter is selected from the group consisting of: an veg promoter, lepA promoter, serA promoter, ymdA promoter, fba promoter, aprE promoter, amyQ promoter, amyL promoter, bacteriophage SPO1 promoter, cryIIIA promoter, combinations thereof, and active fragments or variants thereof. Even more preferably, the promoter sequence is selected from the group consisting of aprE promoter, amyL promoter, veg promoter, bacterio-phage SPO1 promoter, and cryIIIA promoter, and combinations thereof, or active fragments or variants thereof. Still even more preferably, the promoter is selected from the group consisting of: an aprE promoter, SPO1 promoter, such as PE4, PE5, or P15 (preferably as described in WO15118126), tandem promoter comprising the promoter sequences amyl and amyQ (preferably as described in WO9943835), and triple promoter comprising the promoter sequences amyL, amyQ, and cryIIIa (preferably as described in WO2005098016). Most preferably, the promoter is an aprE promoter, preferably, an aprE promoter from Bacillus amyloliquefaciens, Bacillus clausii, Bacillus haloduans, Bacillus lentus, Bacillus licheniformis, Bacillus pumilus, Bacillus subtilis, or Bacillus velezensis, more preferably from Bacillus licheniformis, Bacillus pumilus or Bacillus subtilis, most preferably, from Bacillus licheniformis.

Utilizing an inducer-independent promoter as specified herein above may be advantageous as it allows for continuous expression of the gene of interest throughout the fermentation resulting in a continuous and stable protein production without the need of an inducer molecule. Hence, utilizing an inducer-independent promoter may contribute to improve the yield of the protein of interest. Further, utilizing an inducer-independent promoter as specified herein above may be advantageous as there is no need for an additional feed line for inducer addition, hence it offers a simpler and more robust technical set up for the production line.

It will be understood that the activity of the promoter used in accordance with the method of the present invention, preferably, is not dependent on heat-inducible elements. Accordingly, the promoter to be used as an expression control sequence in accordance of the present invention, preferably, may be a temperature-insensitive promoter and/or lacks a heat-inducible element.

In contrast, thereto an “inducer-dependent promoter” is understood herein as a promoter that is increased in its activity to enable transcription of the gene to which the promoter is operably linked upon addition of an “inducer molecule” to the fermentation medium. Thus, for an inducer-dependent promoter the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule the presence of which in the fermentation medium is capable of affecting an increase in expression of a gene by increasing the activity of an inducer-dependent promoter operably linked to the gene. Inducer molecules known in the art include carbohydrates or analogs thereof that may function as secondary carbon source in addition to a primary carbon source such as glucose. Typically, the Bacillus host cell has not been genetically modified in its ability to take up or metabolize an inducer molecule.

The “protein of interest” as referred to herein refers to any protein, peptide or fragment thereof which is intended to be produced in the Bacillus host cell. A protein, thus, encompasses polypeptides, peptides, fragments thereof as well as fusion proteins and the like.

Preferably, the protein of interest is an enzyme. In particular, the protein of interest is an enzyme that is prone to undergo proteolytic degradation during fermentation or has autoproteolytic activity such as any enzyme catalyzing proteolytic reactions. More particularly, the enzyme is classified as an oxidoreductase (EC 1), a transferase (EC 2), a hydrolase (EC 3), a lyase (EC 4), an isomerase (EC 5), or a ligase (EC 6) (EC-numbering according to Enzyme Nomenclature, Recommendations (1992) of the Nomenclature Committee of the International Union of Biochemistry and Molecular Biology including its supplements published 1993-1999). Preferably, the protein of interest is an enzyme suitable to be used in detergents.

Typically, the protein of interest is a protein that precipitates at a concentration of at least 50 mM of sulfate or of a similar precipitant in the fermentation medium, more typically protein of interest precipitates at a concentration of at least 100 mM, at least 150 mM, at least 200 mM, at least 250 mM, at least 300 mM, at least 350 mM or at least 400 mM of sulfate or of a similar precipitant. More typically, the protein of interest is an enzyme as specified elsewhere herein, that precipitates as described above; even more typically, the protein of interest is a protease that precipitates at a concentration of at least 50 mM of sulfate or of a similar precipitant, more typically protein of interest precipitates at a concentration of at least 100 mM, at 150 mM or at 200 mM of sulfate or of a similar precipitant in the fermentation medium.

Proteins of interest that precipitate already at a lower concentration of sulfate or a similar precipitant may be unfavorable as they might not be easily resolved during subsequent purification processes.

Typically, the protein of interest precipitates from the fermentation medium starting from a concentration of 1 g/L, 2 g/L, 3 g/L, 4 g/L, 5 g/L, 6 g/L, 7 g/L, 8 g/L, 9 g/L, 10 g/L, 12.5 g/L, 15 g/L, 17.5 g/L, 20 g/L, 22.5 g/L, 25 g/L, 27.5 g/L, 30 g/L, 32.5 g/L, 35 g/L, 37.5 g/L, 40 g/L or 45 g/L of protein in the fermentation medium based on the measurement of the protein of interest, typically activity measurements of the enzyme of interest within the fermentation medium.

Typically, the pH of the fermentation medium is in between pH 5-9, more preferably in between 6 and 8.5 and most preferably in between 6.8 and 7.8. These pH ranges are advantageous as they contribute to a good growth of the Bacillus host cells and to the secretion of the protein of interest. It is therefore also contemplated that at these conditions a precipitation of the protein of interest can happen. In other words, the precipitation of the protein of interest typically occurs at a pH value within said pH range between pH 5-9, more preferably in between pH 6 and pH 8.5 and most preferably in between pH 6.8 and pH 7.8, in the fermentation medium.

More preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, glucoamylase, nuclease, and cellulase, preferably, amylase or protease, preferably, a protease. Still more preferred, the enzyme is a serine protease (EC 3.4.21), preferably a subtilisin protease (EC3.4.21.62).

Typically, the protein of interest is a protease selected from proteases having an amino acid sequence with at least 80% of sequence identity, such as at least 85% sequence identity, such as at least 95% sequence identity, such as at least 96% sequence identity, such as at least 97% sequence identity, such as at least 98% sequence identity, such as at least 99% sequence identity to any one of SEQ ID NO: 1-13. More typically, the protein of interest is a protease selected from proteases having an amino acid sequence as specified in any of SEQ ID NO: 1-13, or variants thereof. Alternatively, the protein of interest is an amylase selected from amylases having an amino acid sequence with at least 80% of sequence identity, such as at least 85% sequence identity, such as at least 95% sequence identity, such as at least 96% sequence identity, such as at least 97% sequence identity, such as at least 98% sequence identity, such as at least 99% sequence identity to any one of SEQ ID NO: 14-18. More typically, the protein of interest is an amylase selected from amylases having an amino acid sequence as specified in any of SEQ ID NO: 14-18, or variants thereof.

However, it will be understood that variants of those enzymes may occur either naturally, i.e. as allelic variants between different strains or from other species, or artificially generated. Typically, those variants comprise an amino acid sequence which differs from the sequence shown in any one of SEQ ID NOs: 1-18 by at least one amino acid substitution, addition and/or deletion, wherein the enzyme variant comprising said sequence retains essentially the same biological activity as the enzymes comprising an amino acid sequence shown in any one of SEQ ID NOs: 1-18. More typically, variants comprise an amino acid sequence which has at least 80% sequence identity with the amino acid sequence shown in any one of SEQ ID NOs: 1-18 retaining still the desired activity. Preferably, such variants comprise an amino acid sequence which has m % sequence identity with the amino acid sequence shown in any one of SEQ ID NOs 1-18 retaining still the desired activity, wherein m is an integer between 80 and 100 or an integer between 90 and 100. Even more preferably, such variants comprise an amino acid sequence which has at least 85%, at least 90%, at least 91%, at least 92%, at least 93%, at least 94% at least 95%, at least 96%, at least 97%, at least 98% or at least 99% sequence identity with the amino acid sequence shown in any one of SEQ ID NOs 1-18 retaining still the desired activity.

A particularly preferred protein of interest is a protease comprising an amino acid sequence which has at least 85%, at least 90%, at least 91%, at least 92%, at least 93%, at least 94% at least 95%, at least 96%, at least 97%, at least 98% or at least 99% sequence identity to SEQ ID NO: 1, 2, 7 or 8 and comprising compared to SEQ ID NO: 1 at least two additional negative charges in the loop region of the amino acid residues 98 to 104 according to the numbering of SEQ ID NO: 3.

The term “introduction of at least two negative charges” into a particular amino acid sequence refers to the increase of the net charge of the particular amino acid sequence by at least two negative charges. Such increase of the net charge of the particular amino acid sequence by at least two negative charges is achieved by altering the amino acid sequence and can be reached by one or more amino acid sequence alterations selected from the group consisting of substitution, deletion and insertion, preferably by one or more amino acid substitutions. The increase of the net charge of the particular amino acid sequence by at least two negative charges can be achieved by removing positive charges or by introducing negative charges or by combinations thereof. The four amino acids aspartic acid (Asp, D), glutamic acid (Glu, E), lysine (Lys, K), and arginine (Arg, R) have a side chain which can be charged at neutral pH. At pH 7.0, two are negatively charged: aspartic acid (Asp, D) and glutamic acid (Glu, E) (acidic side chains), and two are positively charged: lysine (Lys, K) and arginine (Arg, R) (basic side chains). Thus, the introduction of at least two negative charges in the amino acid sequence can be reached for instance by substituting arginine by glutamic acid, substituting two non-charged leucine residues by two glutamic acid residues, by inserting two aspartic acid residues or by deleting two lysine residues. The introduction of at least two negative charges by modification of the amino acid sequence is evaluated preferably under conditions usually occurring in a washing step, preferably at pH 6-11, preferably at pH 7-9, more preferably at pH 7.5-8.5, further preferred at pH 7.0-8.0, most preferably at pH 7.0 or pH 8.0.

Preferably, the at least two additional negative charges in the loop region of amino acid residues 98 to 104 according to the numbering of SEQ ID NO: 3 are obtained by one or more amino acid alterations selected from the group consisting of substitutions, deletions, and insertions, preferably by substitutions and/or insertion, most preferred substitution.

In a preferred protease the at least two additional negative charges compared to SEQ ID NO: 1 in the loop region of residues 98 to 104 may be caused by one or more amino acid substitutions at amino acid position according the numbering of SEQ ID NO: 3 selected from the group consisting of 98, 99, 100, 101, 102, 103, and 104, preferably at position 101.

Still preferably, the at least two additional negative charges in the loop region of residues 98 to 104 according to the numbering of SEQ ID NO: 3 are obtained by one or more amino acid alterations selected from the group consisting of X99E, X101D and X101E, preferably, D99E, R101D and R101E, most preferably R101E. X typically refers to any amino acid residue at a given position.

Still preferably, the loop sequence 98 to 104 of a preferred protease has compared to SEQ ID NO: 1 two additional negative charges with the following sequence ADGEGAI, ADGDGAI, ADGDGSV, ADGEGSV, AADGSGSV, AADGEGSV, or ASEGEGSV with longer sequences having an insertion in the loop sequence.

Particularly preferred is a protease comprising an amino acid sequence which has at least 85%, at least 90%, at least 91%, at least 92%, at least 93%, at least 94% at least 95%, at least 96%, at least 97%, at least 98% or at least 99% sequence identity to SEQ ID NO: 1, 2, 7 or 8 and comprising a glutamic acid (E) at position 101 according to the numbering of SEQ ID NO: 3.

More preferred is a protease comprising at least 90% sequence identity to SEQ ID NO: 1 and compared to SEQ ID NO: 1 the amino acid substitution R101E or R101D, preferably R101E, according to the numbering of SEQ ID NO: 3.

Also preferred is a protease comprising at least 90% sequence identity to SEQ ID NO: 1 and compared to SEQ ID NO: 1 the amino acid substitution R101E or R101D, preferably R101E, and the amino acid substitutions S3T, V4I, and V205I, according to the numbering of SEQ ID NO: 3.

Also preferred is a protease comprising at least 90% sequence identity to SEQ ID NO: 1 and compared to SEQ ID NO: 1 the amino acid substitution R101E or R101D, preferably R101E, and one, preferably both, of the substitutions selected from the group consisting of S156D and L262E according to the numbering of SEQ ID NO: 3.

Most preferred is a protease according to any one of SEQ ID NO: 2, SEQ ID NO: 7, or SEQ ID NO: 8.

Sequence identity usually is provided as “% sequence identity”. To determine the percent-sequence-identity between two amino acid sequences in a first step a pairwise sequence alignment is generated between those two sequences, wherein the two sequences are aligned over their complete length (i.e., a pairwise global alignment). The alignment is, preferably, generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gap open=10.0, gap extend=0.5 and matrix=EBLOSUM62). The preferred alignment for the purpose of this invention is that alignment, from which the highest sequence identity can be determined. After aligning the two sequences, in a second step, an identity value shall be determined from the alignment. Therefore, according to the present invention the following calculation of percent-identity applies: x %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “% sequence identity”.

Enzyme variants comprising an amino acid sequence having at least one amino acid substitution, addition and/or deletion may, in particular, also comprise conservative mutations which appear to have a minimal effect on protein folding resulting in certain enzyme properties being substantially maintained when compared to the enzyme properties of the parent enzyme. For determination of %-similarity according to this invention the following applies, which is also in accordance with the BLOSUM62 matrix, which is one of the most used amino acids similarity matrix for database searching and sequence alignments: Amino acid A is similar to amino acids S; Amino acid D is similar to amino acids E; N; Amino acid E is similar to amino acids D; K; Q; Amino acid F is similar to amino acids W; Y; Amino acid H is similar to amino acids N; Y; Amino acid I is similar to amino acids L; M; V; Amino acid K is similar to amino acids E; Q; R; Amino acid L is similar to amino acids I; M; V; Amino acid M is similar to amino acids I; L; V; Amino acid N is similar to amino acids D; H; S; Amino acid Q is similar to amino acids E; K; R; Amino acid R is similar to amino acids K; Q; Amino acid S is similar to amino acids A; N; T; Amino acid T is similar to amino acids S; Amino acid V is similar to amino acids I; L; M; Amino acid W is similar to amino acids F; Y; Amino acid Y is similar to amino acids F; H; W.

Conservative amino acid substitutions may occur over the full length of the sequence of a polypeptide sequence of a functional protein such as an enzyme. Therefore, according to the present invention the following calculation of percent-similarity applies: m %-similarity=[(identical residues+similar residues)/length of the alignment region which is showing the respective sequence of this invention over its complete length]*100. Thus, sequence similarity in relation to comparison of two amino acid sequences herein is calculated by dividing the number of identical residues plus the number of similar residues by the length of the alignment region which is showing the respective sequence of this invention over its complete length. This value is multiplied with 100 to give “%-similarity”. Especially, variant enzymes comprising conservative mutations which exhibit at least m %-similarity to the respective parent sequences with m being an integer between 70 and 100, between 80 and 100 or between 90 and 100, preferably, 70, 75, 80, 85, 90, 91, 92, 93, 94, 95, 96, 97, 98 or 99 compared to the full length polypeptide sequence, are expected to have essentially unchanged enzyme properties. Even more preferably, such variants comprise an amino acid sequence which has at least 80%, at least 85%, at least 90%, at least 91%, at least 92%, at least 93%, at least 94% at least 95%, at least 96%, at least 97%, at least 98% or at least 99% sequence similarity with the amino acid sequence shown in any one of SEQ ID NOs 1-18.

Preferably, the protein of interest is secreted into the fermentation medium, typically by the Bacillus host cells. In other words, preferably, the method according to the invention utilizes a secretory expression system. Secretion of the protein of interest into the fermentation medium is advantageous as it allows for a facilitated separation of the protein of interest from the fermentation medium. For secretion of the protein of interest into the fermentation medium the nucleic acid construct may typically comprise a polynucleotide encoding a signal peptide that directs secretion of the protein of interest into the fermentation medium. Various signal peptides are known in the art. These signal peptides are also referred to as “secretory signal” herein. Preferred signal peptides are selected from the group consisting of the signal peptide of the AprE protein from Bacillus subtilis or the signal peptide from the YvcE protein from Bacillus subtilis.

The signal peptide of the AprE protein from Bacillus subtilis or the signal peptide from the YvcE protein from Bacillus subtilis are particularly suitable for secreting enzymes, such as amylases, from Bacillus cells into the fermentation medium. As the YvcE signal peptide is suitable for secreting a wide variety of different enzymes, including amylases and proteases, this signal peptide can be used, preferably in conjunction with the production method according to the present invention described herein.

It will be understood that each of the expression control sequence, nucleic acid sequence encoding the protein of interest and/or the aforementioned further elements may be from the Bacillus host cell or may be from another species, i.e. heterologous with respect to said Bacillus host cell. In particular, the protein of interest may be heterologously expressed, i.e. the protein of interest may be heterologous with respect to said Bacillus host cell.

Typically, the Bacillus host cell comprises an expression construct comprising at least a gene encoding for the protein of interest under the control of a promoter. More typically, the expression construct may be an arrangement of a gene of interest and the expression control sequences and/or further elements including e.g. a secretory signal as specified elsewhere herein. In particular, the expression construct may comprise a secretory signal as specified elsewhere herein, which may specifically lead to secretion of the protein of interest into the fermentation medium. The said gene of interest, expression control sequences and/or further elements, or part thereof, may be native to, i.e., endogenously present in the genome of the Bacillus host cell. Moreover, the term “expression construct” may also be understood as encompassing native expression constructs which have been genetically manipulated, e.g., by genomic editing and/or mutagenesis technologies.

The expression construct may also be an exogenously introduced expression construct. In an exogenously introduced expression construct, the expression control sequence, the gene encoding the protein of interest and/or the further elements may be native with respect to the host cell or may be derived from other species, i.e. be heterologous with respect to the Bacillus host cell. The introduction of the expression construct into a Bacillus host cell can be accomplished in accordance with the present invention by any method known in the art, including, inter alia, well known transformation, transfection, transduction, and conjugation techniques and the like. Preferably, the expression construct exogenously introduced is comprised in a vector, preferably, an expression vector. The expression vector can be, preferably, located outside the chromosomal DNA of the Bacillus host cell, i.e. be present episomally, in one or more copies. However, the expression vector may also preferably be integrated into the chromosomal DNA of the Bacillus cell in one or more copies. The expression vector can be linear or circular. Preferably, the expression vector is a viral vector or a plasmid.

For autonomous replication, the expression vector may further comprise an origin of replication enabling the vector to replicate autonomously in the host cell in question. Bacterial origins of replication include but are not limited to the origins of replication of plasmids pUB110, pC194, pTB19, pAMß1, and pTA1060 permitting replication in Bacillus (Janniere, L., Bruand, C., and Ehrlich, S. D. (1990). Structurally stable Bacillus subtilis cloning vectors. Gene 87, 53-6; Ehrlich, S. D., Bruand, C., Sozhamannan, S., Dabert, P., Gros, M. F., Janniere, L., and Gruss, A. (1991). Plasmid replication and structural stability in Bacillus subtilis. Res. Microbiol. 142, 869-873), and pE194 (Dempsey, L. A. and Dubnau, D. A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436). Yet, the expression vector, preferably, contains one or more selectable markers that permit easy selection of transformed Bacillus host cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO95/23221, where the selectable marker is on a separate vector.

The method according to the present invention comprises a step of inoculating a fermentation medium with a Bacillus host cell comprising a gene encoding a protein of interest under the control of a promoter which shall be described in more detail below. The term “inoculating” as used herein refers to introducing Bacillus host cells into the fermentation medium used for cultivation. Inoculation of the fermentation medium with the Bacillus host cells can be achieved by introducing Bacillus host cells of a pre-culture (starter culture) into the initial fermentation medium. Preferably, the fermentation medium or the initial fermentation medium is inoculated with pre-culture that has been grown under conditions known to the person skilled in the art. The pre-culture can be obtained by cultivating the cells in a pre-culture medium that can be a chemically defined pre-culture medium or a complex pre-culture medium. The pre-culture medium can be the same or different from the fermentation medium used for cultivation in the method of the present invention. The complex pre-culture medium can contain complex nitrogen and/or complex carbon sources. Preferably, the pre-culture used for inoculation is obtained by using a complex culture medium. The pre-culture can be added all or in part to the fermentation medium of the main fermentation, typically to the initial fermentation medium. Preferably, the Bacillus host cells in the pre-culture are actively growing cells, i.e. they are in a stage where the number of cells is increasing. Typically, cells in a pre-culture are upon inoculation of the pre-culture in a lag phase and switch over time to a phase of exponential growth. Preferably, the volume ratio between pre-culture used for inoculation and the fermentation medium for the main fermentation, is between 0.1 and 30% (v/v), more preferably the volume ratio between pre-culture used for inoculation and the initial fermentation medium is between 0.1 and 30% (v/v).

The method according to the invention further comprises a step of cultivating the Bacillus host cell in the fermentation medium under conditions conducive for the growth of the Bacillus host cell and the expression of the protein of interest. The term “cultivating” or “cultivation” as used herein refers to keeping alive and/or propagating Bacillus host cells comprised in a fermentation medium at least for a predetermined time. The term may encompass phases of exponential cell growth as well as phases of stationary growth or other phases of growth, such as controlled growth. Typically, the term “cultivating” or “cultivation” refers to propagating the Bacillus host cells in a fermentation medium as specified elsewhere herein.

The term “fermentation medium” as used herein refers to a water-based solution containing one or more chemical compounds that can support the growth of cells. Preferably, the fermentation medium according to the present invention is a complex fermentation medium or a chemically defined fermentation medium. More preferably, the fermentation medium according to the present invention is a chemically defined fermentation medium. The term “initial fermentation medium” typically refers to the fermentation medium used initially when starting the fermentation by inoculating the medium with the Bacillus host cells, e.g. with the Bacillus cells from the pre-culture specified elsewhere herein. The term “added to the process” or “added to the fermentation process” is to be understood as an addition to the initial fermentation medium or as an addition with a suitable feed solution during the fermentation process. The components in the fermentation medium typically relate to the sum of components or chemical compounds such as nutrients or other media components that are initially present, i.e. present in the “initial fermentation medium”, and those that are added to the fermentation medium during the fermentation process, e.g. in the feed solution and/or pH adjustment solution and/or the change due to the metabolic activity of the cells.

A complex fermentation medium as used herein refers to a fermentation medium that comprises a complex nutrient source in an amount of 0.5 to 30% (w/v) of the fermentation medium. Complex nutrient sources are nutrient sources which are composed of chemically undefined compounds, i.e., compounds that are not known by their chemical formula, preferably comprising undefined organic nitrogen- and/or carbon-containing compounds. In contrast thereto, a “chemically defined nutrient source” (e.g., “chemically defined carbon source” or “chemically defined nitrogen source”) is understood to be used for nutrient sources which are composed of chemically defined compounds. A chemically defined component is a component which is known by its chemical formula. A complex nitrogen source is a nutrient source that is composed of one or more chemically undefined nitrogen containing compounds, i.e., nitrogen containing compounds that are not known by their chemical formula, preferably comprising organic nitrogen containing compounds, e.g., proteins and/or amino acids with unknown composition. A complex carbon source is a carbon source that is composed of one or more chemically undefined carbon containing compounds, i.e., carbon containing compounds that are not known by their chemical formula, preferably comprising organic carbon containing compounds, e.g., carbohydrates with unknown composition. It is clear for the skilled person that a complex nutrient source might be a mixture of different complex nutrient sources. Thus, a complex nitrogen source can comprise a complex carbon source and vice versa and a complex nitrogen source can be metabolized by the cells in a way that it functions as carbon source and vice versa.

The complex nutrient source may be a complex nitrogen source. Complex sources of nitrogen include, but are not limited to protein-containing substances, such as an extract from microbial, animal or plant cells, e.g., plant protein preparations, soy meal, corn meal, pea meal, corn gluten, cotton meal, peanut meal, potato meal, meat, casein, gelatins, whey, fish meal, yeast protein, yeast extract, tryptone, peptone, bacto-tryptone, bacto-peptone, wastes from the processing of microbial cells, plants, meat or animal bodies, and combinations thereof. In one embodiment, the complex nitrogen source is selected from the group consisting of plant protein,
preferably potato protein, soy protein, corn protein, peanut, cotton protein, and/or pea protein, casein, tryptone, peptone and yeast extract and combinations thereof.

Preferably, the fermentation medium may also comprise defined media components. Preferably, the fermentation medium also comprises a defined nitrogen source. Examples of inorganic nitrogen sources are ammonium, nitrate, and nitrite, and combinations thereof. In a preferred embodiment, the fermentation medium comprises a nitrogen source, wherein the nitrogen source is a complex or a defined nitrogen source or a combination thereof. In one embodiment, the defined nitrogen source is selected from the group consisting of ammonia, ammonium, ammonium salts, (e.g., ammonium chloride, ammonium nitrate, ammonium phosphate, ammonium sulfate, ammonium acetate), urea, nitrate, nitrate salts, nitrite, and amino acids, preferably, glutamate, and combinations thereof.

Preferably, the complex nutrient source is in an amount of 2 to 15% (v/w) of the fermentation medium. In another embodiment, the complex nutrient source is in an amount of 3 to 10% (v/w) of the fermentation medium.

Also preferably, the complex fermentation medium may further comprise a carbon source. The carbon source is, preferably, a complex or a defined carbon source or a combination thereof. Preferably, the complex nutrient source comprises a carbohydrate source. Various sugars and sugar-containing substances are suitable sources of carbon, and the sugars may be present in different stages of polymerization. Preferred complex carbon sources to be used in the present invention are selected from the group consisting of molasses, corn steep liquor, cane sugar, dextrin, starch, starch hydrolysate, and cellulose hydrolysate, and combinations thereof. Preferred defined carbon sources are selected from the group consisting of carbohydrates, organic acids, and alcohols, preferably, glucose, fructose, galactose, xylose, arabinose, sucrose, maltose, lactose, acetic acid, propionic acid, lactic acid, formic acid, malic acid, citric acid, fumaric acid, glycerol, inositol, mannitol and sorbitol, and combinations thereof. Preferably, the defined carbon source is provided in form of a syrup, which can comprise up to 20%, preferably, up to 10%, more preferably up to 5% impurities. In one embodiment, the carbon source is sugar beet syrup, sugar cane syrup, corn syrup, preferably, high fructose corn syrup. In another embodiment, the complex carbon source is selected from the group consisting of molasses, corn steep liquor, dextrin, and starch, or combinations thereof, and wherein the defined carbon source is selected from the group consisting of glucose, fructose, galactose, xylose, arabinose, sucrose, maltose, dextrin, lactose, or combinations thereof.

Preferably, the fermentation medium is a complex medium comprising complex nitrogen and complex carbon sources. More preferably, the fermentation medium is a complex medium comprising complex nitrogen and carbon sources, wherein the complex nitrogen source may be partially hydrolyzed as described in WO 2004/003216.

Yet, the fermentation medium may, typically, also comprises a hydrogen source, an oxygen source, a sulfur source, a phosphorus source, a magnesium source, a sodium source, a potassium source, a trace element source, and a vitamin source as further described elsewhere herein.

Typically, the fermentation medium used in the method according to the present invention is a chemically defined fermentation medium. A chemically defined fermentation medium is a fermentation medium which is essentially composed of chemically defined components in known concentrations. A chemically defined component is a component which is known by its chemical formula. A fermentation medium which is essentially composed of chemically defined component includes a medium which does not contain a complex nutrient source, in particular, no complex carbon and/or complex nitrogen source, i.e., which does not contain complex raw materials having a chemically undefined composition. A fermentation medium which is essentially composed of chemically defined components may further include a medium which comprises an essentially small amount of a complex nutrient source, for instance a complex nitrogen and/or carbon source, an amount as defined below, which typically is not sufficient to maintain growth of the Bacillus host cells and/or to guarantee formation of a sufficient amount of biomass.

In that regard, complex raw materials have a chemically undefined composition due to the fact that, for instance, these raw materials contain many different compounds, among which complex heteropolymeric compounds, and have a variable composition due to seasonal variation and differences in geographical origin. Typical examples of complex raw materials functioning as a complex carbon and/or nitrogen source in fermentation are soybean meal, cotton seed meal, corn steep liquor, yeast extract, casein hydrolysate, molasses, and the like. An essentially small amount of a complex carbon and/or nitrogen source may be present in the chemically defined fermentation medium according to the invention, for instance as carry-over from the inoculum for the main fermentation. The inoculum for the main fermentation is not necessarily obtained by fermentation on a chemically defined medium. Most often, carry-over from the inoculum will be detectable through the presence of a small amount of a complex nitrogen source in the chemically defined fermentation medium of the main fermentation. Small amounts of a complex medium components, like complex carbon and/or nitrogen source, might also be introduced into the fermentation medium by the addition of small amounts of these complex components to the fermentation medium. It may be advantageous to use a complex carbon and/or nitrogen source in the fermentation process of the inoculum for the main fermentation, for instance to speed up the formation of biomass, i.e. to increase the growth rate of the microorganism, and/or to facilitate internal pH control. For the same reason, it may be advantageous to add an essentially small amount of a complex carbon and/or nitrogen source, e.g. yeast extract, to the initial stage of the main fermentation, especially to speed up biomass formation in the early stage of the fermentation process. An essentially small amount of a complex nutrient source which may be added to the chemically defined fermentation medium in the fermentation process according to the invention is defined to be an amount of at the most 10% of the total amount of the respective nutrient, which is added in the fermentation process. In particular, an essentially small amount of a complex carbon and/or nitrogen source which may be added to the chemically defined fermentation medium is defined to be an amount of a complex carbon source resulting in at the most 10% of the total amount of carbon and/or an amount of a complex nitrogen source resulting in at the most 10% of the total amount of nitrogen, which is added in the fermentation process, preferably an amount of a complex carbon source resulting in at the most 5% of the total amount of carbon and/or an amount of a complex nitrogen source resulting in at the most 5% of the total amount of nitrogen, more preferably an amount of a complex carbon source resulting in at the most 1% of the total amount of carbon and/or an amount of a complex nitrogen source resulting in at the most 1% of the total amount of nitrogen, which is added in the fermentation process. Preferably, at the most 10% of the total amount of carbon and/or at the most 10% of the total amount of nitrogen, preferably an amount of at the most 5% of the total amount of carbon and/or an amount of at the most 5% of the total amount of nitrogen, more preferably an amount of at the most 1% of the total amount of carbon and/or an amount of at the most 1% of the total amount of nitrogen which is added in the fermentation process is added via carry-over from the inoculum. Most preferably, no complex carbon and/or complex nitrogen source is added to the fermentation medium in the fermentation process.

A chemically defined nutrient source as referred to herein e.g., chemically defined carbon source or chemically defined nitrogen source, is understood to be used for nutrient sources which are composed of chemically defined compounds.

Culturing a microorganism in a chemically defined fermentation medium requires that cells be cultured in a medium which contain various chemically defined nutrient sources selected from the group consisting of chemically defined hydrogen source, chemically defined oxygen source, chemically defined carbon source, chemically defined nitrogen source, chemically defined sulfur source, chemically defined phosphorus source, chemically defined magnesium source, chemically defined sodium source, chemically defined potassium source, chemically defined trace element source, and chemically defined vitamin source. Preferably, the chemically defined carbon source is selected from the group consisting of carbohydrates, organic acids, hydrocarbons, alcohols and mixtures thereof. Preferred carbohydrates are selected from the group consisting of glucose, fructose, galactose, xylose, arabinose, sucrose, maltose, maltotriose, lactose, dextrin, maltodextrins, starch and inulin, and mixtures thereof. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Preferably, the chemically defined carbon source comprises glucose or sucrose. More preferably, the chemically defined carbon source comprises glucose, even more preferably the predominant amount of the chemically defined carbon source is provided as glucose.

Most preferably, the chemically defined carbon source is glucose. It is to be understood that the chemically defined carbon source can be provided in form of a syrup, preferably as glucose syrup. As understood herein, glucose as referred to herein shall include glucose syrups. A glucose syrup is a viscous sugar solution with high sugar concentration. The sugars in glucose syrup are mainly glucose and to a minor extent also maltose and maltotriose in varying concentrations depending on the quality grade of the syrup. Preferably, besides glucose, maltose and maltotriose the syrup can comprise up to 10%, preferably, up to 5%, more preferably up to 3% impurities. Preferably, the glucose syrup is from corn.

The chemically defined nitrogen source is preferably selected from the group consisting of urea, ammonia, nitrate, nitrate salts, nitrite, ammonium salts such as ammonium chloride, ammonium sulfate, ammonium acetate, ammonium phosphate and ammonium nitrate, and amino acids such as glutamate or lysine and combinations thereof. More preferably, a chemically defined nitrogen source is selected from the group consisting of ammonia, ammonium sulfate and ammonium phosphate. Most preferably, the chemically defined nitrogen source is ammonia. The use of ammonia as a chemically defined nitrogen source has the advantage that ammonia additionally can function as a pH controlling agent.

Additional compounds can be added in complex and chemically defined fermentation medium as described below.

Oxygen is usually provided during the cultivation of the cells by aeration of the fermentation media by stirring and/or gassing. Hydrogen is usually provided due to the presence of water in the aqueous fermentation medium. However, hydrogen and oxygen are also contained within the carbon and/or nitrogen source and can be provided that way.

Magnesium can be provided to the fermentation medium by one or more magnesium salts, preferably selected from the group consisting of magnesium chloride, magnesium sulfate, magnesium nitrate, magnesium phosphate, and combinations thereof, or by magnesium hydroxide, or by combinations of one or more magnesium salts and magnesium hydroxide.

Sodium can be added to the fermentation medium by one or more sodium salts, preferably selected from the group consisting of sodium chloride, sodium nitrate, sodium sulfate, sodium phosphate, sodium hydroxide, and combinations thereof.

Calcium can be added to the fermentation medium by one or more calcium salts, preferably selected from the group consisting of calcium sulfate, calcium chloride, calcium nitrate, calcium phosphate, calcium hydroxide, and combinations thereof.

Potassium can be added to the fermentation medium in chemically defined form by one or more potassium salts, preferably selected from the group consisting of potassium chloride, potassium nitrate, potassium sulfate, potassium phosphate, potassium hydroxide, and combinations thereof.

Phosphorus can be added to the fermentation medium by one or more salts comprising phosphorus, preferably selected from the group consisting of potassium phosphate, sodium phosphate, magnesium phosphate, phosphoric acid, and combinations thereof. Preferably, at least 1 g of phosphorus is added per liter of initial fermentation medium.

Sulfur is added to the fermentation medium by one or more salts comprising sulfur, preferably selected from the group consisting of ammonium sulfate, potassium sulfate, sodium sulfate, magnesium sulfate, sulfuric acid, and combinations thereof.

In the method according to the invention, sulfate is added to the fermentation medium to reach a concentration of at least 20 mM of sulfate in the fermentation medium. Preferably, the sulfate is added with the feed to reach or to maintain the desired amount. Alternatively, the sulfate is already present in the desired amount in the initial fermentation medium or added thereto at the start of the fermentation or at least added to the fermentation medium at the start of the production phase of the protein of interest. Even more preferred, the sulfate is present in a high amount in the initial fermentation medium and added with the feed during the fermentation process such that the desired sulfate concentration is maintained or reached.

In particular, the amount of sulfate added to the initial fermentation medium reaches a concentration of at least 25 mM, at least 50 mM, at least 100 mM, at least 150, at least 200, at least 250 mM, at least 300 mM, or at least 400 mM of sulfate. More particularly, the amount of sulfate added to the fermentation medium reaches a concentration of at least 25 mM, at least 50 mM, at least 100 mM, at least 150, at least 200, at least 250 mM, at least 300 mM, at least 400 mM, at least 500 mM, or at least 600 mM of sulfate in the fermentation medium. These concentrations reflect the amount of sulfate or other precipitants that is desired according to the present invention in other words, these concentrations reflect the desired amount. Typically the concentration does not exceed 1 M of sulfate in the fermentation medium. A preferred concentration of sulfate in the fermentation medium is in the range of 100 mM to 750 mM, more preferred between 150 mM and 700 mM, still more preferred between 200 mM and 600 mM, still more preferred between 300 mM and 550 mM,

More particularly, the sulfate may be added to the fermentation medium as a feed solution during the fermentation process. Even more particularly, the sulfate concentration may reach its maximum during the main fermentation. At the end of the fermentation process, e.g. prior to the step of obtaining the protein of interest, the sulfate concentration is preferably below the maximum. This way of sulfate feeding may be advantageous as it avoids the undesired carry-over of sulfate into subsequent process steps such as steps of purifying the protein of interest.

Typically and advantageously, the amount of sulfate added to the fermentation medium is sufficient to achieve precipitation and/or crystallization of the protein of interest.

Other precipitation agents or crystallization aids may be used as an alternative or in addition to the herein mentioned sulfate. Typical precipitation agents include alkaline metal salts and earth alkaline metal salts, and the corresponding ammonium salts thereof, preferably the precipitation agents include phosphate and citrate salts, halogenide salts and acetates of the afore mentioned alkaline, earth alkaline metals and ammonium. Typical crystallization aids include organic solvents such as methanol, ethanol, isopropanol, acetone, dimethyl formamide and the like. Other typical precipitation agents include polyethylene glycol of different molecular weights such as PEG400 or PEG800 or PEG 4000. It is understood that precipitation can also be performed by using also a mixture of different precipitation agents.

In line with the present invention, the sulfate may be added to the fermentation medium in any suitable form of a sulfate containing compound including sulfate containing salts or acids. The sulfate compound may be added as a liquid or in solid form. Typically, the sulfate is selected from the group of sulfate containing compounds consisting of (NH₄)₂SO₄, MgSO₄, K₂SO₄, Na₂SO₄, sulfuric acid and combinations thereof, preferably sulfuric acid or Na₂SO₄. Typically, the addition of the sulfate containing compound may include a base with the conjugated acid, e.g. adding sodium hydroxide and sulfuric acid.

Preferably, the sulfate is added to the fermentation medium as feed solution during cultivation and/or is present in the initial fermentation medium. More preferably, the sulfate is present in the initial fermentation medium, typically in an amount of at least 20 mM, more typically in an amount of at least 25 mM, at least 50 mM, at least 100 mM, at least 150, at least 200, at least 250 mM, at least 300 mM, or at least 400 mM of sulfate. Particularly preferred is an amount of sulfate in a range between 100 mM and 350 mM in the initial fermentation medium, still particularly preferred is a range between 150 mM and 300 mM of sulfate in the initial fermentation medium. In addition or alternatively, the sulfate may be added to the fermentation medium as a feed solution such as described elsewhere herein. Most preferably, the sulfate is present in the initial fermentation medium and is added to the fermentation medium as a feed solution during cultivation, e.g. during the fermentation process. Even more preferably, sulfate is added during the fermentation process to the fermentation medium, for example with the feed, in order to reach a desired concentration of at least 100 mM, typically at least 150 mM, at least 200 mM, at least 250 mM, at least 300 mM, at least 400 mM, or at least 500 mM of sulfate in the fermentation medium. In particular once the desired concentration is reached, it is maintained throughout the main fermentation process. Preferably, in the step of adding sulfate to the fermentation medium sulfate is added to reach a concentration in the range of 100 mM to 750 mM, more preferred in the range of 200 mM to 600 mM, even more preferred in the range of 300 mM to 550 mM of sulfate in the fermentation medium.

Typically, if the desired sulfate concentration is not present in the initial fermentation medium, it is reached within the first 60 h of the fermentation process, such as within 60 h after inoculating the initial fermentation medium with the Bacillus host cell. More typically, in such a scenario, i.e. if the desired sulfate concentration is not present in the initial fermentation medium, the sulfate concentration may typically be increased with the feed up to the desired concentration in the fermentation medium. Still more typically, the desired sulfate concentration is reached within the first 50 h, 40 h, 30 h, or 20 h of the fermentation process.

The desired sulfate concentration may preferably be maintained for at least 40 h up to 120 h. More preferably, the desired sulfate concentration may preferably be maintained for at least 48 h up to 100 h during the fermentation process.

Typically, the desired concentration of sulfate is maintained starting with the enzyme titer equivalent to 10%, 20%, or 30% of the maximum achievable titer of the protein of interest, wherein the titer of the protein of interest is defined as the sum of soluble and precipitated protein of interest.

Advantageously, the inventors found that the claimed high concentration of sulfate in the fermentation medium, in particular at the beginning of the fermentation process, has advantageous effects on the production of the protein of interest such as an increase in productivity and/or yield of the protein of interest. Without being bound by theory, the present inventors believe that the high amounts of sulfate induce precipitation and/or crystallization of the protein of interest. This even applies to proteins of interest that have a strong tendency to auto-proteolysis and/or proteins of interest that under common production processes do not form crystals, in particular under common production processes that do not have the desired sulfate amounts, such as processes that have lower than the desired amount of sulfate in the fermentation medium. Thereby, auto-proteolytic activity of the protein of interest is kept at a minimum. Further, proteolytic or hydrolytic damage to the protein of interest caused by host cell enzymes or unfavorable conditions in the fermentation medium is kept at a minimum. Thus, the precipitation and/or crystallization of the protein of interest is thought to stabilize the protein of interest during the fermentation process and results in an increase in productivity and yield.

Although not required, protein seed crystals may be added in an amount up to about 10% by weight, based on the weight of the solution, to promote the crystallization process. The use of seed crystals makes the kinetics of the crystallization more favorable and seems to increase overall yield of crystalline protein. Nucleation may also be promoted by providing crystallization vessels having surface properties conducive to crystallization, which properties are well known by persons skilled in the art. The use of the minimum but effective amount of seed crystals for a given protein solution, size of operation, process conditions, etc., will be apparent to one skilled in the art, as in conventional crystallization processes.

The method according to the invention is in particularly advantageous for producing a protein of interest that is prone to undergo proteolysis by host proteases, i.e. enzymes such as amylase, lipase, mannanase, phytase, xylanase, phosphatase, glucoamylase, nuclease, and cellulose; or for producing a protein of interest that is prone to undergo auto-proteolysis such as proteases. More particularly the method according to the invention is advantageous for a protein of interest that has not been genetically engineered for reducing solubility for example by enhancing its precipitation and/or crystallization capacity.

Preferably, the fermentation medium and/or the initial fermentation medium, comprises one or more selected from the group consisting of:
0.1 to 50 g nitrogen per liter of fermentation medium;
1 to 6 g phosphorus per liter of fermentation medium;
0.15 to 2 g sulfur per liter of fermentation medium;
0.4 to 8 g potassium per liter of fermentation medium;
0.01 to 2 g sodium per liter of fermentation medium;
0.01 to 3 g calcium per liter of fermentation medium; and
0.1 to 10 g magnesium per liter of fermentation medium.

According to the present invention, the fermentation medium may comprise an amino acid derivative in an amount of 0-30 g/l of fermentation medium.

Preferably, the fermentation medium comprises an amino acid derivative in an amount of 0.005-30 g/L of fermentation medium, more preferably an amount of 0.005-12 g/L of fermentation medium. The amino acid derivative may alternatively or additionally be present in the feed solution in an amount of 0.0125-30 g/L of the feed solution.

If present, the amino acid derivative may typically be present in the fermentation medium in an amount of at least 5 mg/L; more typically the amino acid derivative is present in the fermentation medium in an amount of 0.005 g/L-12 g/L. Even more typically, the amino acid derivative is present in an amount of 0.05 g/L to 10 g/L, still even more typically, in an amount of 0.5 g/L to 5 g/L.

The amount of amino acid derivative present in the fermentation medium may typically depend on the Bacillus host cell used or may be independent thereof. In case the Bacillus host cell is a Bacillus licheniformis cell, there may be no addition of amino acid derivative into the fermentation medium, hence typically then the amount of amino acid derivative in the fermentation medium may be 0 mg/L of fermentation medium. In other words, in case the Bacillus host cell is a Bacillus licheniformis cell the amino acid derivative may be absent from the fermentation medium and the feed solution, and be absent from the fermentation medium.

In case the Bacillus host cell is a Bacillus licheniformis cell, the amount of the amino acid derivative added to the process may be 0 mg/l of fermentation medium, hence there may be no addition of the amino acid derivative to the fermentation medium. Hence, for a Bacillus licheniformis host cell, the fermentation medium may not comprise any substantial amount of an amino acid derivative. However, even in case when the Bacillus host cell is a Bacillus licheniformis cell, the amount of amino acid derivative may be at least 0.5 mg/L of the fermentation medium, or at least 2 mg/L of the fermentation medium. Particularly preferred are amounts of betaine in the range of 0.5 to 10 mg/L. It was found by the present inventors that a combined addition of high sulfate and betaine has advantageous effects on the productivity in Bacillus host cells.

Alternatively, when the Bacillus host cell is a Bacillus subtilis cell, the amount of the amino acid derivative may be at least 10 mg/L of fermentation medium.

An “amino acid derivative” according to the present invention refers to an amino acid compound or a compound related thereto. These compounds may commonly be referred to as osmoprotectants. Preferably, the amino acid compound is selected from the group consisting of betaine, choline, proline, and ectoine, preferably betaine. The term betaine preferably is understood as referring to trimethylglycine.

Alternatively, the fermentation medium may comprise a quaternary ammonium compound such as a compound selected from betaine and choline.

Typically, the method according to the invention comprises a step of adding the amino acid derivative to the fermentation medium. More typically, the amino acid derivative is added to the fermentation medium as feed solution during cultivation and is present in the initial fermentation medium. However, the amino acid derivative may also be added to the fermentation medium as feed solution during cultivation or be present in the initial fermentation medium.

Adding the specified amount of an amino acid derivative to the fermentation medium comprising a suitably high amount of sulfate may be advantageous as it may contribute to further enhancing growth of the Bacillus host cell under the described conditions designed to limit autoproteolytic processes and/or to avoid instability or reduced activity of the protein of interest. Without wishing to be bound by theory the inventors of the present invention assume that the specific combination of the described addition of the amino acid derivative with a suitable amount of sulfate leads to a significantly enhanced production of the protein of interest. This is also shown in the example given below.

Typically, the feed solution differs from the fermentation medium and/or from the initial fermentation medium, in one or more of the compounds of said group listed above. Even more typically, the feed solution differs from the fermentation medium and/or from the initial fermentation medium, in the amount of one or more of the compounds of said group listed above.

One or more trace element ions can be added to the fermentation medium, preferably in amounts of below 10 mmol/L initial fermentation medium each. These trace element ions are selected from the group consisting of iron, copper, manganese, zinc, cobalt, nickel, molybdenum, selenium, and boron and combinations thereof. Preferably, the trace element ions iron, copper, manganese, zinc, cobalt, nickel, and molybdenum are added to the fermentation medium. Preferably, the one or more trace element ions are added to the fermentation medium in an amount selected from the group consisting of 50 μmol to 5 mmol per liter of initial medium of iron, 40 μmol to 4 mmol per liter of initial medium copper, 30 μmol to 3 mmol per liter of initial medium manganese, 20 μmol to 2 mmol per liter of initial medium zinc, 1 μmol to 100 μmol per liter of initial medium cobalt, 2 μmol to 200 μmol per liter of initial medium nickel, and 0.3 μmol to 30 μmol per liter of initial medium molybdenum, and combinations thereof. For adding each trace element preferably one or more from the group consisting of chloride, phosphate, sulfate, nitrate, citrate and acetate salts can be used.

Compounds which may optionally be included in the fermentation medium are chelating agents, such as citric acid, MGDA, NTA, or GLDA, and buffering agents such as mono- and dipotassium phosphate, calcium carbonate, and the like. Buffering agents preferably are added when dealing with processes without an external pH control. In addition, an antifoaming agent may be dosed prior to and/or during the fermentation process.

Vitamins refer to a group of structurally unrelated organic compounds, which are necessary for the normal metabolism of cells. Cells are known to vary widely in their ability to synthesize the vitamins they require. A vitamin should be added to the fermentation medium of Bacillus cells not capable of synthesizing said vitamin. Vitamins can be selected from the group of thiamin, riboflavin, pyridoxal, nicotinic acid or nicotinamide, pantothenic acid, cyanocobalamin, folic acid, biotin, lipoic acid, purines, pyrimidines, inositol, and hemins.

Preferably, the fermentation medium also comprises a selection agent, e.g., an antibiotic, such as ampicillin, tetracycline, kanamycin, hygromycin, bleomycin, chloramphenicol, streptomycin or phleomycin, to which the selectable marker of the cells provides resistance.

The amount of necessary compounds to be added to the medium will mainly depend on the amount of biomass which is to be formed in the fermentation process. The amount of biomass formed may vary widely, typically the amount of biomass is from about 10 to about 150 grams of dry cell mass per liter of fermentation medium. Usually, for protein production, fermentations producing an amount of biomass which is lower than about 10 g of dry cell mass per liter of fermentation medium are not considered industrially relevant.

The optimum amount of each component of a defined medium, as well as which compounds are essential and which are non-essential, will depend on the type of Bacillus cell which is subjected to fermentation in a medium, on the amount of biomass and on the product to be formed. Typically, the amount of medium components necessary for growth of the microbial cell may be determined in relation to the amount of carbon source used in the fermentation, typically in relation to the main carbon source, since the amount of biomass formed will be primarily determined by the amount of carbon source used. Particular preferred fermentation media are also described in the Example below.

Preferably, the fermentation medium is sterilized prior to use in order to prevent or reduce growth of microorganisms during the fermentation process, which are different from the inoculated microbial cells. Sterilization can be performed with methods known in the art, for example but not limited to, autoclaving or sterile filtration. Some or all medium components can be sterilized separately from other medium components to avoid interactions of medium components during sterilization treatment or to avoid decomposition of medium components under sterilization conditions.

In the method according to the invention, the Bacillus host cell is cultivated in the fermentation medium under conditions conducive for the growth of the Bacillus host cell and the expression of the protein of interest. The phrase “conditions conducive for the growth of the Bacillus host cell and the expression of the protein of interest” means that conditions other than the temperature or fermentation medium used for cultivation. Such conditions comprise pH during cultivation, physical movement of the culture by shaking or stirring and/or atmospheric conditions applied to the culture.

The pH of the fermentation medium during cultivation may be adjusted or maintained. Preferably, the pH of the medium is adjusted prior to inoculation. Preferred pH values envisaged for the fermentation medium are within the range of about pH 6.6 to about pH 9, preferably within the range of about pH 6.6 to about pH 8.5, more preferably within the range of about pH 6.8 to about pH 8.5, most preferably within the range of about pH 6.8 to about pH 8.0. As an example, for a Bacillus cell host cell culture, the pH is, preferably, adjusted to or above about pH 6.8, about pH 7.0, about pH 7.2, about pH 7.4, or about pH 7.6. Preferably, the pH of the fermentation medium during cultivation of the Bacillus host cell culture is adjusted to a pH within the range of about pH 6.8 to about pH 9, preferably about pH 6.8 to about pH 8.5, more preferably about pH 7.0 to about pH 8.5, most preferably about pH 7.0 to about pH 8.0.

Physical movement can be applied by stirring and/or shaking of the fermentation medium. Suitable techniques for achieving physical movement during the cultivation of Bacillus host cells in a fermentation medium are well known in the art and can be applied by the skilled artisan without further ado. Besides stirring, oxygen or other gases may be applied to the culture by adjusting suitable atmospheric conditions. Preferably, oxygen is supplied with 0 to 3 bar air or oxygen.

Furthermore, additional conditions including the selection of suitable bioreactors or vessels for cultivation of Bacillus host cells are well known in the art and can be made by the skilled artisan without further ado.

The term “feed solution” as used herein refers to a solution that is added to the fermentation medium after inoculation of the initial fermentation medium with Bacillus host cells. The initial fermentation medium typically refers to the fermentation medium present in the fermenter at the time of inoculation with the Bacillus host cells. The feed solution comprises compounds supportive for the growth of said cells. Compared to the fermentation medium the feed solution may be enriched for one or more compounds.

A feed medium or feed solution used e.g. when the culture is run in fed-batch mode may be any of the above mentioned medium components or combination thereof. It is understood herein that at least part of the compounds that are provided as feed solution can already be present to a certain extent in the fermentation medium prior to feeding of said compounds.

Preferably, said feed solution provides a primary carbon source comprising at least one carbohydrate, typically during the cultivation phase. Alternatively said feed solution provides a primary carbon source comprising at least one carbohydrate in a first cultivation phase and/or in one or more subsequent cultivation phase(s). More preferably, the carbohydrate comprised in the feed solution represents the main source of carbon consumed or metabolized by the host cell. Still more preferably, the feed solution comprises a chemically defined carbon source, even more preferably, glucose. Even more preferably, the feed solution comprises 40% to 75% glucose, preferably 42% to 70% glucose, more preferably 45% to 67% glucose, even more preferably 50% to 65% glucose. Even more preferably, glucose is the main carbon source present in the feed solution and/or in the fermentation medium. Typically, the same feed solution may be used for the seed fermenter run in fedbatch mode and the production bioreactor. The feed solution used for the seed fermenter run in fedbatch mode may differ from the feed solution used in the production bioreactor. However, the feed solution used for the seed fermenter run in fedbatch mode and the feed solution used in the production bioreactor may have the same concentration of glucose, but the feed solution used in the production bioreactor contains salts which are not present in the feed solution used for the seed fermenter run in fedbatch mode.

A feed solution can be added continuously or discontinuously during the fermentation process. Discontinuous addition of a feed solution can occur once during the fermentation process as a single bolus or several times with different or same volumes. Continuous addition of a feed solution can occur during the fermentation process at the same or at varying rates (i.e., volume per time). Also combinations of continuous and discontinuous feeding profiles can be applied during the fermentation process. Components of the fermentation medium that are provided as feed solution can be added in one feed solution or as different feed solutions. In case more than one feed solution is applied, the feed solutions can have the same or different feed profiles as described above. Particular preferred feed solutions are also described in the Examples below.

In line with the present invention, the feed solution may comprise the amino acid derivative in an amount of 0.0125-30 g/l of the feed solution.

Typically, the main carbon source may be provided to the fermentation medium in a feed solution different from the feed solution comprising further nutrients. In other words, typically at least two different feed solutions may be added into the fermentation medium during the fermentation process, more typically at least two different feed solution differing in their composition may be added into the fermentation medium during the fermentation process. Still even more typically one feed solution comprises the main carbon source, and the other feed solution comprises further nutrients such as salts and/or sulfate.

The phrase “precipitation and/or crystallization of the protein of interest” as used herein refers to the formation of any kind of insoluble particles of the protein of interest or comprising the protein of interest in the fermentation medium. These particles may be in a crystalline form, in the form of an amorphous precipitate or in the form of an aggregate, or a mixture thereof. Precipitation and/or crystallization of the protein of interest may typically be achieved by the addition of sulfate to the fermentation medium as specified elsewhere herein. More typically, the protein of interest is secreted to the outside of the Bacillus host cell into the fermentation medium and, after being secreted into the fermentation medium, precipitates, crystallizes or aggregates. Alternatively, the protein of interest is present as an insoluble particle in the Bacillus host cell before it is released into the fermentation medium. Precipitation and/or crystallization of the protein of interest typically occurs at a concentration of at least 20 mM of sulfate in the fermentation medium or of a precipitant as defined elsewhere herein. More typically precipitation and/or crystallization of the protein of interest occurs at a concentration of at least 50 mM, at least 100 mM, at least 150 mM, at least 200 mM, at least 250 mM, at least 300 mM, at least 350 mM, at least 400 mM, or at least 450 mM of sulfate or of a similar precipitant in the fermentation medium. Although Precipitation and/or crystallization of the protein of interest may occur at the above described concentration of sulfate, using a higher sulfate concentration in the desired range exceeding said concentration at which the precipitation and/or crystallization protein of interest occurs is beneficial as it may increase the amount and/or the speed of the precipitation and/or crystallization process.

Specifically, the method according to the invention has a step of adding sulfate to the fermentation medium to reach a concentration of sulfate in the fermentation medium sufficient for allowing or inducing the protein of interest of interest to precipitate and/or crystallize during cultivation.

The precipitation and/or crystallization of the protein of interest in the fermentation medium may further be enhanced by the addition of flocculating agents. Typical flocculating agents are known in the art and include cationic, anionic and/or non-ionic polyacrylamides, polyethyleneimine (PEI), poly(diallyldimethylammonium chloride) (pDADMAC), polyamines, natural polymers from microorganisms, e.g. alginates, chitosan, gelatin and others, and chemical flocculants such as soluble Fe or Al compounds disclosed in WO 96/38469. The type and amount of the flocculating agents are selected based on the properties of the particular selected fermentation medium and will typically be determined using simple routine experimentation. This it known in the art and the selection of type and amount of one or more flocculating agents to facilitate the separation is completely within the skills of the skilled practitioner. For example, may the methods disclosed in WO 2004/001054 be used according to the present invention.

The step “allowing the protein of interest to precipitate and/or crystallize during cultivation” refers to a step wherein the precipitation and/or crystallization of the protein of interest occurs. This is typically achieved by the addition of sulfate in a sufficient amount or typically in the described desired amount and may further depend on the protein of interest. Hence, the addition of the desired amount of sulfate specifically induces the precipitation and/or crystallization of the protein of interest. Moreover, the pH of the fermentation medium may be adjusted to enhance precipitation and/or crystallization of the protein of interest. Still further, protein seed crystals may be added, typically from a solution containing protein crystals, in an amount up to about 10% by weight, based on the weight of the solution, to additionally promote the crystallization process.

Preferably, the method according to the invention includes a step of obtaining the protein of interest. The phrase “obtaining the protein of interest” as used herein typically refers to obtaining a product such as the protein of interest from the Bacillus host cell culture by appropriate purification techniques including techniques known in the art and including those described elsewhere herein. More preferably, the step of obtaining the protein of interest comprises a step of purifying the protein of interest, preferably by separating the liquid fraction and the solid fraction of the fermentation medium, thereby obtaining the protein of interest at least partially in the solid fraction.

Solid/liquid separation techniques are known in the art and include techniques such as centrifugation, filtration, or settling followed by decanting. These may be typically used in the method according to the invention, in particular in the step of separating the liquid fraction and the solid fraction of the fermentation medium.

In particular said solid/liquid separation techniques result in at least a fraction comprising the liquid part of the fermentation medium, typically referred to as “liquid fraction”, and at least a solid fraction comprising the solid parts present in the fermentation medium. Preferably, at least the solid fraction of the fermentation medium comprises the precipitate and/or crystals of the protein of interest. However, the liquid fraction may comprise at least a part of the protein of interest, in particular the part being solubilized in the fermentation medium.

The term “solid fraction of the fermentation medium” denotes the fraction of a fermentation medium obtained after solid/liquid separation, including cells, insoluble substrates, and insoluble fermentation products such as precipitated an or crystallized protein of interest.

More typically, the step of purifying the protein of interest comprises
(i) separating the fermentation medium in a first phase and a second phase, wherein the first phase comprises the liquid fraction, the protein of interest in soluble form and optionally cells and cell debris, and the second phase, the solid fraction, comprises the protein of interest in precipitated form, cells and cell debris; and
(ii) optionally solubilizing the precipitated or crystallized form of the protein of interest in the second phase (solid fraction).

For purifying the protein of interest, step (i) may be performed prior to step (ii). Alternatively, an additional step of solubilizing or dissolving the precipitated or crystallized form of the protein of interest present in the fermentation medium may be performed prior to step (i).

Hence, following the step of separating the solid and the liquid fraction, or prior to the step of separating the solid and the liquid fraction, the method according to the invention may comprise dissolving or solubilizing the protein of interest present in crystallized or precipitated form. In particular, the protein of interest may be dissolved or solubilized by at least one of the following steps:
dissolving the solid fraction in a suitable solvent;
adding a compound promoting solubilization such as divalent soluble salt of calcium, magnesium, iron, zinc; and/or
adjusting the pH.

However, performing the step of separating the solid and the liquid fraction e.g. as in step (i), prior to solubilizing or dissolving the precipitated or crystallized form of the protein of interest e.g. as in step (ii), may be advantageous as it requires only small amounts of suitable solvent, solubilization promoting compound and/or pH adjusting agent.

The divalent salt for promoting solubilization may preferably be a phosphate, sulfate, acetate, nitrate, or chloride salt.

The pH may be adjusted for dissolving the protein of interest depending on the kind of protein of interest. For example, in the case the protein of interest is amylase, the pH is typically adjusted to a high value, such as a value in the range of 9.0-12.0. In case the protein of interest is a protease, the pH is typically adjusted to a low value such as a range of 1.5 to 6.0, more typically to a value in the range of 3.0 to 5.5. Even more typically, the pH may be adjusted to a pH value below 7.0, 6.0, below 5.5, below 5.0, or below 4.5 but above 3.0.

Methods for dissolving or solubilizing proteins in solid form, in particular in precipitated and/or crystalized form are known in the art and disclosed in for example EP 2125865; U.S. Pat. No. 3,316,240 and WO 93/13125. These methods are also suitable for use according to the present invention. The terms “solubilizing” and “dissolving” are used interchangeably herein.

In the method according to the invention, the solubilization step may be performed by diluting the solid fraction with a suitable solvent, e.g. with water or an aqueous media. Further optionally adding a solubilization promoting compound and adjusting the pH. Typically, the solubilization may be done by diluting the second fraction comprising the protein of interest in solid form 100-2000% (w/w), preferably 100-1000% (w/w), based on the amount of the solid fraction and typically the concentration of the precipitant.

The solvent may typically be water or an aqueous medium. The pH adjustment may be done before, simultaneously or after the addition of the divalent salt. Further steps may be included such as a mixing may take place. The mixing time will typically depend on the chosen temperature and the crystal morphology and/or structure of the protein of interest in question. More than 80% of the crystals and/or precipitate of the protein of interest, and/or protein of interest bound to cell mass and/or insolubles may be solubilized according to the present invention; preferably more than 85%; more preferably more than 90% and in particular more than 95% of the crystals and/or precipitate of the protein of interest, and/or protein of interest bound to cell mass and/or insolubles may be solubilized.

Moreover, solubilization of the protein of interest may be performed by use of a polyol, such as low molecular weight polyethylene glycol and the C₂ through C₈ alcohols having at least two OH groups, preferably with only two OH groups, especially preferred is polyols where two OH groups are present on adjacent carbon atoms in the chain, and the C₂-C₈ alcohol is aliphatic and have a straight carbon chain.

Preferred polyols include ethylene glycol, propylene glycol, mono-propylene glycol, glycerol, the low molecular weight (about 900 or less) polyethylene glycols, and mixtures thereof.

Moreover, the method of the invention may further comprise a second separation step removing cells and cell debris from the mixture comprising the solubilized protein of interest. This second separation may typically be a further solid/liquid separation as explained elsewhere herein.

The method according to the invention may further comprise one or more of the following additional purification steps e.g. for recovering the protein of interest: ultracentrifugation, evaporation, centrifugation, spray draying, granulation, freeze-drying, stabilization, and the like. These purification steps and methods are known in the art and may be adapted to the present invention by way of routine experimentation.

Further, the method according to the present invention may comprise a step of adding one or more stabilizers and/or adding one or more protease inhibitors. These additives are known in the art and may serve to stabilize the protein of interest. Moreover, the method according to the present invention may comprise a step of preparing a formulation containing the protein of interest. “Protein formulation” means any non-complex formulation comprising a small number of ingredients, wherein the ingredients serve the purpose of stabilizing the protein, in particular the protein of interest, comprised in the protein formulation and/or the stabilization of the protein formulation itself. The term “protein stability” relates to the retention of proteins activity as a function of time during storage or operation. The term “protein formulation stability” relates to the maintenance of physical appearance of the protein formulation during storage or operation as well as the avoidance of bacterial contamination during storage or operation.

The protein formulation can be either solid or liquid. Protein formulations can be obtained by using techniques known in the art. For instance, without being limited thereto, solid enzyme formulations can be obtained by extrusion or granulation. Suitable extrusion and granulation techniques are known in the art and are described for instance in WO 94/19444 A1 and WO 97/43482 A1. The protein of interest obtained in the method according to the invention may optionally be concentrated by partially removing water. The water can be removed by ways known to the skilled artisan such as evaporation, spray drying or spray granulation.

Moreover, the step of purifying the protein of interest may optionally comprise one or more pretreatment steps, typically prior to solid/liquid separation, These pretreatment steps may include processes such as dilution, adjusting pH and/or temperature in the fermentation medium, adding stabilizers capable of preventing further growth of the Bacillus host cell to the fermentation medium, adding inhibitors capable of reducing protease activity and thereby limiting degradation due to proteolytic activity. The pretreatment may also comprise a lysis step e.g. treating the broth with a chemical lysing agent such as one or more cell wall degrading enzymes e.g. lysozyme(s). Preferred lysis methods include adding lysozyme and/or hydrolyzing enzymes to solubilize suspended material as cells or residual fermentation raw materials.

The present invention further contemplates the use of a high sulfate concentration in a fermentation medium for producing a protein of interest in a Bacillus host cell. The terms “high sulfate concentration” and “desired sulfate concentration” are used interchangeably herein. Typically, a high sulfate concentration refers to an amount of sulfate of at least 20 mM, preferably to at least 25 mM, at least 50 mM, at least 100 mM, at least 150, at least 200, at least 250 mM, at least 300 mM, at least 400 mM, or at least 500 mM of sulfate. Typically, the sulfate concentration does not exceed a concentration of at most 10 M, at most 5 M, at most 2 M, at most 1000 mM, at most 800 mM or at most 600 mM in the fermentation medium.

Moreover, the present invention contemplates the use of a combination of sulfate and an amino acid derivative for producing a protein of interest in Bacillus host cell in a fermentation medium.

Moreover, the present invention relates to a precipitated or, crystallized protein of interest obtained by or obtainable by the method according to the invention.

Advantageously, it has been found that the method in accordance with the present invention provides an improved way of producing a protein of interest that may be instable or undergo auto-proteolysis under conventional industrial fermentation processes. The claimed method in particular provides means for producing stabilized protein of interest with high productivity and high yield reducing production costs to an attractive level. The method achieves a high productivity as the total amount of protein of interest produced per reactor volume per hour of process time is improved compared to prior art processes superior. The method is suitable for large scale industrial fermentation processes and is cost-efficient as there is no need for specialized equipment or expensive specialty chemicals. Moreover, the method according to the invention is suitable for various kinds of proteins of interest, in particular proteases, and for various Bacillus host cell strains.

The explanations and interpretations of the terms given above in this specification apply for all embodiments characterized herein. The following embodiments are particular preferred embodiments according to the present invention.

One embodiment relates to a method for producing a protein of interest in a fermentation medium comprising the following steps:
a) inoculating a fermentation medium with a Bacillus host cell comprising a gene encoding a protein of interest under the control of a promoter;
b) cultivating the Bacillus host cell in the fermentation medium under conditions conducive for the growth of the Bacillus host cell and the expression of the protein of interest,
c) adding sulfate to the fermentation medium to reach a concentration of at least 20 mM of sulfate in the fermentation medium; and
d) allowing the protein of interest to precipitate and/or crystallize during cultivation;
   wherein the fermentation medium comprises an amino acid derivative in an amount of 0-30 g/L of fermentation medium.

Further embodiments are:
1. A method for producing a protein of interest in a fermentation medium comprising the following steps:
   a) inoculating a fermentation medium with a Bacillus host cell comprising a gene encoding a protein of interest under the control of a promoter;
   b) cultivating the Bacillus host cell in the fermentation medium under conditions conducive for the growth of the Bacillus host cell and the expression of the protein of interest,
   c) adding sulfate to the fermentation medium to reach a concentration of at least 20 mM of sulfate in the fermentation medium; and
   d) allowing the protein of interest to precipitate and/or crystallize during cultivation.
2. The method according to embodiment 1, wherein the Bacillus host cell is a cell of Bacillus amyloliquefaciens, Bacillus clausii, Bacillus haloduans, Bacillus lentus, Bacillus licheniformis, Bacillus pumilus, Bacillus subtilis, or Bacillus velezensis, preferably Bacillus licheniformis.
3. The method according to any one of the preceding embodiments, wherein in step c) sulfate is added to reach a concentration in a range of 100 mM to 750 mM of sulfate in the fermentation medium, preferably in a range of 200 mM to 600 mM of sulfate in the fermentation medium.
4. The method according to any one of the preceding embodiments, wherein the desired sulfate concentration is reached within the first 50 h, 40 h, 30 h, or 20 h of the fermentation process.
5. The method according to any one of the preceding embodiments, wherein the protein of interest is secreted into the fermentation medium.
6. The method according to any one of the preceding embodiments, including the following step:
   e) obtaining the protein of interest.
7. The method according to the preceding embodiment 6, wherein the method further comprises a step of purifying the protein of interest, preferably by separating the liquid fraction and the solid fraction of the fermentation medium, thereby obtaining the protein of interest at least partially in the solid fraction.
8. The method according to any one of the preceding embodiments, wherein the fermentation medium is a chemically defined fermentation medium.
9. The method according to any one of the preceding embodiments, wherein the protein of interest is heterologously expressed, preferably wherein the protein of interest is an enzyme, more preferably wherein the protein of interest is selected from the group consisting of: amylase, protease, lipase, mannanase, phytase, xylanase, phosphatase, glucoamylase, nuclease, and cellulase, more preferably selected from amylase, protease and mannanase; even more preferably the protein of interest is a protease.
10. The method according to any one of the preceding embodiments, wherein the protein of interest is a protease selected from proteases having an amino acid sequence with at least 80% of sequence identity, such as at least 85% sequence identity, such as at least 95% sequence identity, such as at least 96% sequence identity, such as at least 97% sequence identity, such as at least 98% sequence identity, such as at least 99% sequence identity to any one of SEQ ID NO: 1-13, or the protein of interest is an amylase selected from amylases having an amino acid sequence with at least 80% of sequence identity, such as at least 85% sequence identity, such as at least 95% sequence identity, such as at least 96% sequence identity, such as at least 97% sequence identity, such as at least 98% sequence identity, such as at least 99% sequence identity to any one of SEQ ID NO: 14-18.
11. The method according to any one of the preceding embodiments, wherein the sulfate is present in the initial fermentation medium.
12. The method according to any one of the preceding embodiments, wherein the initial fermentation medium comprises at least 20 mM of sulfate, more typically at least 25 mM, at least 50 mM, at least 100 mM, at least 150, at least 200, at least 250 mM, at least 300 mM, or at least 400 mM of sulfate.
13. The method according to any one of the preceding embodiments, wherein the sulfate is added to the fermentation medium as feed solution during cultivation.
14. The method according to any one of the preceding embodiments, wherein the sulfate is selected from the group of sulfate containing compounds consisting of (NH₄)₂SO₄, MgSO₄, K₂SO₄, Na₂SO₄, sulfuric acid and combinations thereof, preferably sulfuric acid or Na₂SO₄.
15. The method according to any one of the preceding embodiments, wherein the amino acid derivative is provided to the fermentation medium as feed solution during cultivation and/or is present in the initial fermentation medium.
16. The method according to any one of the preceding embodiments, wherein the amino acid derivative is selected from the group consisting of betaine, choline, proline, and ectoine, preferably betaine, more preferably trimethylglycine.
17. The method according to any one of the preceding embodiments, wherein the fermentation medium comprises an amino acid derivative in an amount of 0.005-12 g/l of fermentation medium.
18. The method according to any one of the preceding embodiments, wherein the amino acid derivative is present in the feed solution in an amount of 0.0125-30 g/l of the feed solution.
19. The method according to any one of the preceding embodiments, wherein the Bacillus host cell comprises an expression construct comprising at least the gene encoding for a protein of interest under the control of a promoter.
20. The method of the preceding embodiment, wherein the expression construct comprises a secretory signal.
21. The method according to the preceding embodiment 7, wherein the step of separating the liquid fraction and the solid fraction of the fermentation medium comprises centrifugation filtration, or settling followed by decanting.
22. The method according to the preceding embodiment 7, wherein the step of purifying the protein of interest comprises:
   (i) separating the fermentation medium in a first phase and a second phase, wherein the first phase comprises the liquid fraction, the protein of interest in soluble form and optionally cells and cell debris, and the second phase, the solid fraction comprises the protein of interest in precipitated form, cells and cell debris; and
   (ii) solubilizing the precipitated form of the protein of interest in the second phase (solid fraction).
23. The method according to the preceding embodiment, wherein the protein of interest comprised in the solid fraction is dissolved, preferably by at least one of the following steps:
   dissolving the solid fraction in a suitable solvent;
   adding a compound promoting solubilization such as divalent soluble salt of magnesium, iron, zinc; and/or
   adjusting the pH.
24. The method according to embodiment 22, wherein the solubilization is performed by use of a polyol, such as low molecular weight polyethylene glycol and the C₂ through C₈ alcohols having at least two OH groups, preferably with only two OH groups, especially preferred is polyols where two OH groups are present on adjacent carbon atoms in the chain, and the C₂-C₈ alcohol is aliphatic and have a straight carbon chain.
25. The method according to embodiment 23, wherein the divalent salt is a phosphate, sulfate, acetate, nitrate, or chloride salt.
26. The method according to the preceding embodiments 23-26, wherein the pH is adjusted to a pH value in the range of 1.5 to 6.0.
27. The method according to any one of the preceding embodiments 23-27, wherein the pH is adjusted to a pH value in the range of 3.0 to 5.5.
28. The method according to any one of the preceding embodiments, further comprising one or more of the following additional purification steps: ultracentrifugation, evaporation, centrifugation, spray draying, granulation, freeze-drying, stabilization, and the like.
29. The method according to any one of the preceding embodiments, further comprising a step of preparing a formulation containing the protein of interest.
30 The method according to any one of the preceding embodiments, further comprising a step of adding one or more stabilizers and/or adding one or more protease inhibitors.
31. The method according to any one of the preceding embodiments, wherein when the Bacillus host cell is a Bacillus licheniformis cell, the amount of the amino acid derivative added to the process is 0 mg/L of fermentation medium.
32. The method according to any one of the preceding embodiments, wherein when the Bacillus host cell is a Bacillus subtilis cell, the amount of the amino acid derivative is at least 10 mg/l of the fermentation medium.
33. The method according to embodiment 30 or 32, wherein the protein of interest is a protease selected from any one of SEQ ID NO: 2, 7, or 8.
34. The method according to any one of the preceding embodiments, wherein the Bacillus host cell is a Bacillus licheniformis cell, the amount of the amino acid derivative is at least 0.5 mg/L of the fermentation medium, and the protein of interest is a protease selected from any one of SEQ ID NO: 2, 7, or 8.
35. Use of a high sulfate concentration in a fermentation medium for producing a protein of interest in a Bacillus host cell.
36. Use according to the preceding embodiment, wherein a high sulfate concentration refers to an amount of sulfate of at least 20 mM, preferably to at least 25 mM, at least 50 mM, at least 100 mM, at least 150, at least 200, at least 250 mM, at least 300 mM, at least 400 mM, or at least 500 mM of sulfate in the fermentation medium.
37. Use of a combination of a sulfate and an amino acid derivative for producing a protein of interest in Bacillus host cell in a fermentation medium.
38. A crystallized protein of interest obtained by or obtainable by the method of any of the preceding embodiments.

All references are herewith incorporated by reference in their entireties as well as with respect to the disclosure content specifically mentioned in this specification.

### Sequences

SEQ ID NOs: 1-13 correspond to protease sequences.

SEQ ID NOs: 14-18 correspond to amylase sequences.

SEQ ID NOs: 19-25 correspond to protease loop sequences.

SEQ ID NO: 26 corresponds to the sequence of the protease substrate peptide.

### FIGURES

FIG. 1 shows the normalized productivity for the control fermentation without SO₄ and betaine addition (open circles) and for the fermentation with SO₄ and betaine addition (solid circles)

FIG. 2 shows the normalized productivity for the control fermentation without SO₄ and betaine addition (open circles), for the fermentation with SO₄ addition (diamonds) and for the fermentation with SO₄ and betaine addition (triangles).

FIG. 3 shows the normalized productivity for the control fermentation without betaine addition (open circles) and for the fermentation with betaine addition (diamonds).

FIG. 4: 4A shows the normalized product titer for the control fermentation (open circles) and for the fermentation experiment according the invention (diamonds). 4B shows the fraction of soluble protease relative to the total amount of protease for the control fermentation (open circles) and for the experiment fermentation (diamonds).

### EXAMPLES

The Examples shall merely illustrate the invention. They shall by no means be construed as limiting the scope.

### Example 1: Improved Productivity of Alkaline Protease 1 by Addition of Sulfate and Betaine

A Bacillus licheniformis strain comprising a gene expressing an alkaline protease as specified in SEQ ID NO: 2 and as described for example in WO95/23221 was cultivated in a fermentation process using a chemically defined fermentation medium providing the components listed in Table 1 and Table 2:

TABLE 1
Macroelements and trace element solution provided
in the fermentation process to the control fermentation
and fermentation according to the invention

| | | Concentration |
|---|---|---|
| Compound | Formula | [g/L initial volume] |
| Citric acid | C₆H₈O₇ | 17 |
| Calcium sulfate dihydrate | CaSO₄ × 2H₂O | 1 |
| Monopotassium phosphate | KH₂PO₄ | 35 |
| Magnesium sulfate heptahydrate | MgSO₄*7H₂O | 12 |
| Trace element solution | | 7 |
| (see Table 3) | | |

TABLE 2
Sulfate and betaine added to the fermentation
medium during the fermentation

| | | Concentration |
|---|---|---|
| Compound | Formula | [g/L initial volume] |
| Sodium sulfate | Na₂SO₄ | 85 |
| Betaine monohydrate | C₅H₁₁NO₂ × H₂O | 3 |

In the process according to the invention the sulfate concentration in the initial fermentation medium was sodium sulfate 27 g/L and for betaine monohydrate 3 g/L.

TABLE 3
Trace element composition of the trace element
solution comprising 40 g/L citric acid

| | Concentration |
|---|---|
| Compound | [μM initial volume] |
| Manganese (Mn²⁺) | 24 |
| Zinc (Zn²⁺) | 17 |
| Copper (Cu⁺) | 32 |
| Cobalt (Co²⁺) | 1 |
| Nickel (Ni²⁺) | 2 |
| Molybdenum (Mb⁴⁺) | 0.2 |

### Iron (Fe²⁺) 38

The fermentation was started with a medium containing 8 g/L glucose. Two conditions were tested: a control fermentation without sodium sulfate and betaine addition and an experimental fermentation with addition of 85 g/L sodium sulfate and 3 g/L of betaine monohydrate. A solution containing 50% glucose was used as feed solution. In Table 1, the total amount of elements summed up together in the fermentation medium referring to the initial fermentation volume, i.e. in the initial fermentation medium and the feed, is given. The amount of sulfate and betaine added to the fermentation medium in the experimental fermentation is specified in Table 2. In both experiments, the total amount of added glucose was above 200 g per liter of initial medium in accordance to the requirements of industrially relevant fermentation processes. The pH was controlled above 7 during fermentation using ammonia. The cultivation temperature was controlled at 30° C.

### Measurement of Protease Titer

The titer of the produced protease for the fermentation process was determined at various time points. Proteolytic activity was determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring OD405.

### Result

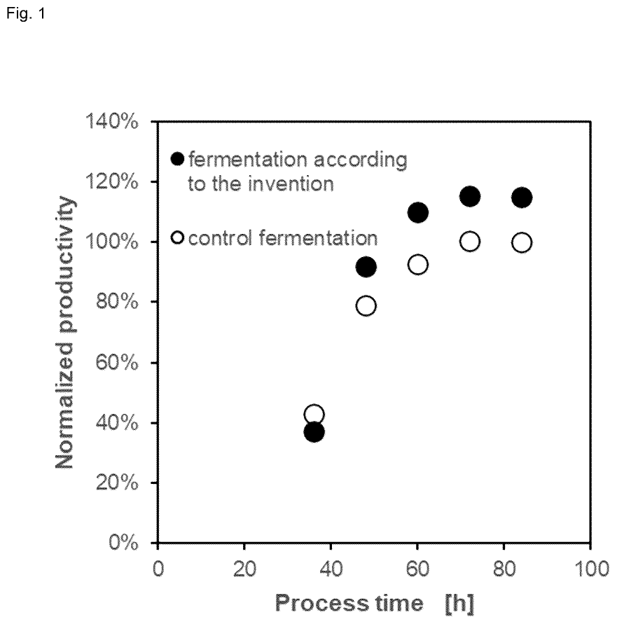

The results are depicted in FIG. 1 showing the development of the normalized productivity over time. Productivity is defined as the total amount of protease produced per reactor volume per hour of process time. The final productivity of the control fermentation was used as the 100% value for the normalization. In both fermentations the productivity increases over the process time and reaches a plateau in the late phase of the process. The fermentation with addition of sulfate and betaine (fermentation according to the invention) outperforms the control fermentation without addition of sulfate and betaine with an increase in productivity by 15%. Thus, adding sulfate and betaine to the process increased the amount of protein of interest produced by the Bacillus host cell.

### Example 2: Improved Productivity of Alkaline Protease 1 by Addition of Sulfate and Betaine

A Bacillus licheniformis strain comprising a gene expressing an alkaline protease as specified in SEQ ID NO: 2 and as described in WO95/23221 was cultivated in a fermentation process using a chemically defined fermentation medium providing the components listed in Table 1 and Table 4:

TABLE 4
Sulfate and betaine added to the fermentation
medium in the experimental fermentations

| Compound | | Concentration |
|---|---|---|
| volume] | Formula | [g/L per initial |
| Sodium sulfate | Na₂SO₄ | 95 |
| Betaine monohydrate | C₅H₁₁NO₂ × H₂O | 3 * |

* only added in the second experimental fermentation

In the second experimental fermentation process the sulfate concentration in the initial fermentation medium was sodium sulfate 38 g/L and for betaine monohydrate 3 g/L.

The fermentation was started with a medium containing 8 g/L glucose. Three conditions were tested: a control fermentation without sodium sulfate and without betaine addition, an experimental fermentation with addition of 95 g/L sodium sulfate per initial volume and a second experimental fermentation with addition of 95 g/L sodium sulfate per initial volume and 3 g/L of betaine monohydrate per initial volume. A solution containing 50% glucose was used as feed solution. In Table 1, the total amount of elements summed up together in the fermentation medium referring to the initial fermentation volume, i.e. in the initial fermentation medium and the feed, is given inn Table 4,

In both experiments, the total amount of added glucose was above 200 g per liter of initial medium in accordance to the requirements of industrially relevant fermentation processes. The pH was controlled above 7 during fermentation using ammonia. The cultivation temperature was controlled at 30° C. Cultivation time was 96 h.

### Measurement of Protease Titer

The titer of the produced protease for the fermentation process was determined at various time points. Proteolytic activity was determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring OD405.

### Result

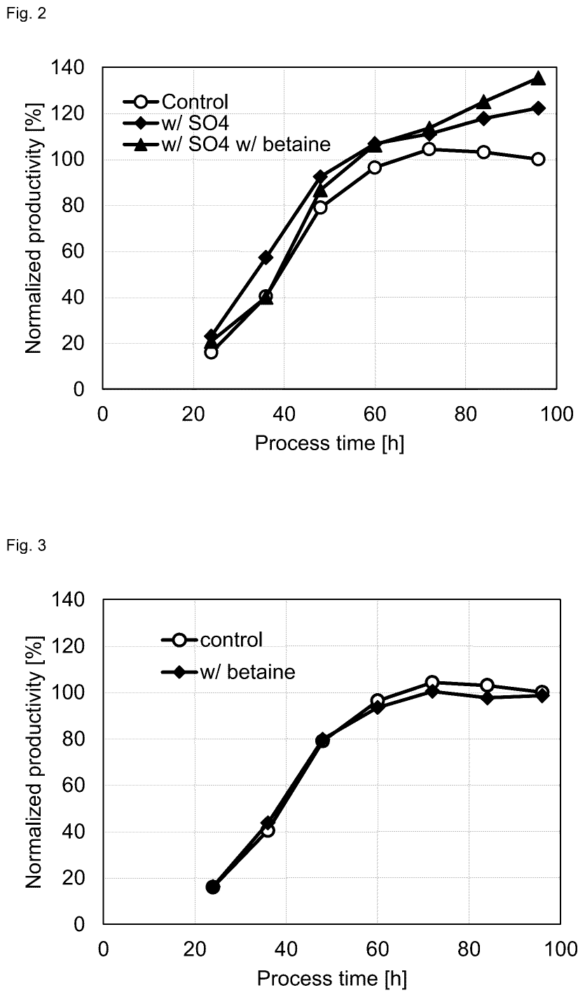

The results are depicted in FIG. 2 showing the development of the normalized productivity over time. Productivity is defined as the total amount of protease produced per reactor volume per hour of process time. The final productivity of the control fermentation was used as the 100% value for the normalization. In all fermentations the productivity increases over the process time and reaches a plateau in the late phase of the process. The fermentation with addition of sulfate but without betaine outperforms the control fermentation without addition of sulfate and betaine with an increase in productivity by 22%. The fermentation with addition of sulfate and betaine (fermentation according to the invention) outperforms the control fermentation with an increase in productivity by 35%. Thus, adding sulfate and betaine to the process increased the amount of protein of interest produced by the Bacillus host cell compared to the control fermentation and compared to a fermentation where only sulfate is added.

### Example 3: Addition of Betaine Alone does not Improve Productivity of Alkaline Protease 1

The Bacillus strain from example 1 was cultivated according to the control fermentation described in example 1. In a second fermentation, 3 g/L per initial volume of betaine monohydrate 3 g/L were added.

### Measurement of Protease Titer

Measurement of protease titer was performed as described in example 1.

### Result

The results are depicted in FIG. 3 showing the development of the normalized productivity over time. Productivity is defined as the total amount of protease produced per reactor volume per hour of process time. The final productivity of the control fermentation was used as the 100% value for the normalization. In both fermentations the productivity increases over the process time and reaches a plateau in the late phase of the process. The fermentation with addition of betaine does not show any improvement in productivity compared to the control fermentation. Thus, adding betaine to the process did not increase the amount of protein of interest produced by the Bacillus host cell compared to the control fermentation.

### Example 4: Providing the Required Precipitant Concentration at an Early Stage of Protein Production Improves Productivity of Alkaline Protease 1

A Bacillus licheniformis strain comprising a gene expressing an alkaline protease as specified in SEQ ID NO: 2 and as described in WO95/23221 was cultivated in a fermentation process using a chemically defined fermentation medium providing the components listed in Table 5 and Table 3:

TABLE 5
Macroelements and trace element solution provided
in the fermentation process to the control fermentation
and fermentation according to the invention

| | | Concentration |
|---|---|---|
| | | [g/L initial |
| Compound | Formula | volume] |
| Citric acid | C₆H₈O₇ | 17 |
| Calcium sulfate dihydrate | CaSO₄ × 2H₂O | 1 |
| Monopotassium phosphate | KH₂PO₄ | 35 |
| Magnesium sulfate heptahydrate | MgSO₄*7H₂O | 12 |
| Trace element solution (see Table 3) | | 7 |
| Sodium sulfate | Na₂SO₄ | 85 |

In the process according to the invention the sulfate concentration in the initial fermentation medium was either sodium sulfate 0 g/L (control experiment) or 45 g/L (fermentation experiment according the invention).

The fermentation was started with a medium containing 8 g/L glucose. Two conditions were compared: achieving the required precipitant concentration as early as possible by already providing said concentration in the initial media (experiment) compared to achieving the required concentration by feeding the precipitant over the course of the fermentation. To test this, two fermentations were performed: a fermentation where sodium sulfate was not added in the initial fermentation medium but added exclusively via the feed to achieve the final concentration stated in table 5 (control). A second fermentation where the sodium sulfate concentration in the initial medium was set to the concentration required to precipitate the protease i.e. 45 g/L, while the rest of the sodium sulfate was added via the feed to achieve the final concentration stated in table 5 (experiment). A solution containing 50% glucose was used as feed solution. In Table 5, the total amount of elements summed up together in the fermentation medium referring to the initial fermentation volume, i.e. in the initial fermentation medium and the feed, is given. In both experiments, the total amount of added glucose was above 200 g per liter of initial medium in accordance to the requirements of industrially relevant fermentation processes. The pH was controlled above 7 during fermentation using ammonia. The cultivation temperature was controlled at 30° C. Cultivation time was 107 h.

### Measurement of Protease Titer

The titer of the produced protease for the fermentation process was determined at various time points. Proteolytic activity was determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring OD405.

### Result

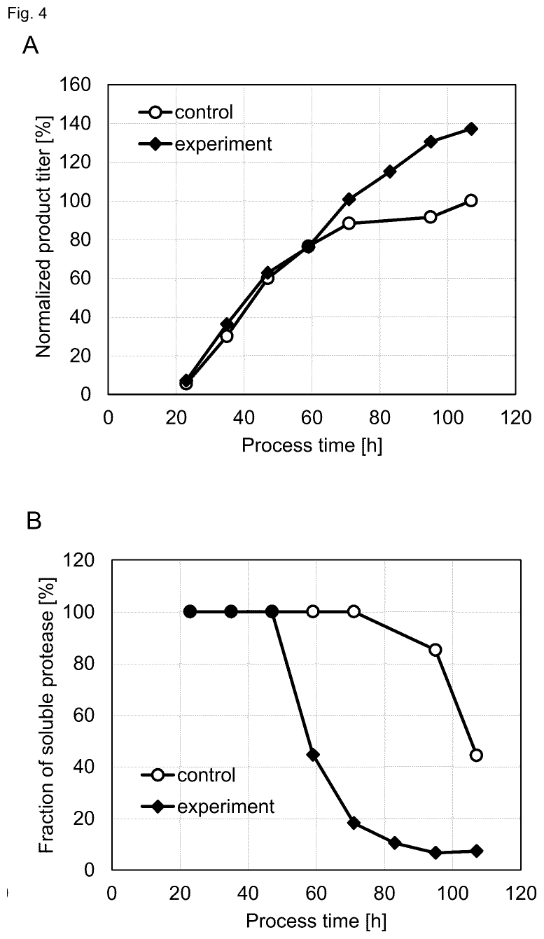

The results are depicted in FIG. 4 showing the development of the normalized product titer over time. Product titer of the control fermentation was used as the 100% value for the normalization. FIG. 4A shows the titer of total protease (soluble and precipitated) and FIG. 4B shows the fraction of soluble protease relative to the total amount of protease for each time point. In both fermentations the product titer increases over the process time. In the fermentation experiment according to the invention the protease precipitates earlier (˜50 h in the fermentation) compared to the control fermentation where precipitation of the protease starts after 70 h. The experiment fermentation resulted in an increase of 37% of the final product titer compared to the control fermentation. This clearly demonstrates that achieving the required precipitant concentration as early as possible by already providing said concentration of precipitant in the initial media and thereby precipitating the protease as early as possible results in increased product titers.

1. A method for producing a protein of interest in a fermentation medium comprising the following steps:
a) inoculating a fermentation medium with a Bacillus host cell comprising a gene encoding a protein of interest under the control of a promoter;
b) cultivating the Bacillus host cell in the fermentation medium under conditions conducive for the growth of the Bacillus host cell and the expression of the protein of interest,
c) adding sulfate to the fermentation medium to reach a concentration of at least 20 mM of sulfate in the fermentation medium; and
d) allowing the protein of interest to precipitate and/or crystallize during cultivation.

2. The method according claim 1, wherein in step c) sulfate is added to reach a concentration in a range of 100 mM to 750 mM of sulfate in the fermentation medium.

3. The method according to claim 1, wherein the sulfate concentration is reached within the first 50 h, 40 h, 30 h, or 20 h of the fermentation process.

4. The method according to claim 1, wherein the protein of interest is secreted into the fermentation medium.

5. The method according to claim 1, further comprising the following step:
e) obtaining the protein of interest.

6. The method according to claim 1, wherein the method further comprises a step of purifying the protein of interest.

7. The method according to claim 1, wherein the protein of interest is heterologously expressed.

8. The method according to claim 1, wherein the protein of interest is a protease selected from proteases having an amino acid sequence with at least 80% of sequence identity, at least 85% sequence identity, at least 95% sequence identity, at least 96% sequence identity, at least 97% sequence identity, at least 98% sequence identity, or at least 99% sequence identity to any one of SEQ ID NO: 1-13, or wherein the protein of interest is an amylase selected from amylases having an amino acid sequence with at least 80% of sequence identity, at least 85% sequence identity, at least 95% sequence identity, at least 96% sequence identity, at least 97% sequence identity, at least 98% sequence identity, or at least 99% sequence identity to any one of SEQ ID NO: 14-18.

9. The method according to claim 1, wherein the sulfate is selected from the group consisting of (NH₄)₂SO₄, MgSO₄, K₂SO₄, Na₂SO₄, sulfuric acid and combinations thereof.

10. The method according to claim 1, wherein the sulfate is present in the initial fermentation medium.

11. The method according to claim 1, wherein the initial fermentation medium comprises at least 20 mM, at least 25 mM, at least 50 mM, at least 100 mM, at least 150, at least 200, at least 250 mM, at least 300 mM, or at least 400 mM of sulfate.

12. The method according to claim 1, wherein the Bacillus host cell comprises an expression construct comprising at least the gene encoding for a protein of interest under the control of a promoter.

13. The method of claim 12, wherein the expression construct comprises a secretory signal.

14. The method according to claim 6, wherein the step of separating the liquid fraction and the solid fraction of the fermentation medium comprises centrifugation filtration, or settling followed by decanting.

15. The method according to claim 6, wherein the protein of interest comprised in the solid fraction is dissolved.

16. (canceled)

17. The method according claim 1, wherein in step c) sulfate is added to reach a concentration in a range of 200 mM to 600 mM of sulfate in the fermentation medium.

18. The method according to claim 1, wherein the step of purifying comprises separating the liquid fraction and the solid fraction of the fermentation medium, thereby obtaining the protein of interest at least partially in the solid fraction.

19. The method according to claim 7, wherein the protein of interest is an enzyme.

20. The method according to claim 19, wherein the enzyme is an amylase, protease, lipase, mannanase, phytase, xylanase, phosphatase, glucoamylase, nuclease, or cellulase.

21. The method according to claim 15, wherein the protein of interest comprised in the solid fraction is dissolved by at least one of the following steps:
resolving the solid fraction in a suitable solvent;
adding a compound promoting solubilization such as divalent soluble salt of magnesium, iron, zinc; and/or
adjusting the pH.
